(19) United States
(12) Patent Application Publication
(10) Pub. No.: US 2025/0376679 A1
(43) Pub. Date: Dec. 11, 2025

(54) METHODS AND COMPOSITIONS FOR INHIBITION OF IRF4

(71) Applicant: The University of North Carolina at Chapel Hill, Chapel Hill, NC (US)

(72) Inventors: Chad Pecot, Pittsboro, NC (US); Christina Ford, Chapel Hill, NC (US); Sascha Tuchman, Durham, NC (US)

(21) Appl. No.: 18/875,271

(22) PCT Filed: Jun. 21, 2023
(86) PCT No.: PCT/US2023/068800

§ 371 Date: Dec. 16, 2024

Related U.S. Application Data
(60) Provisional application No. 63/354,049, filed on Jun. 21, 2022.

(51) Int. Cl. C12N 15/113

(52) U.S. Cl. CPC C12N 15/113; C12N 2310/14; C12N 2310/315; C12N 2310/321; C12N 2310/322; C12N 2310/351

(57) ABSTRACT

The invention relates to the inhibition of expression of interferon regulatory factor-4 (IRF4) using RNA interference, chemically-modified oligonucleotides, and/or chimeric siRNA multivalent combinations. The invention further relates to methods of treating IRF4 related conditions such as multiple myeloma.

### STATEMENT OF PRIORITY

This application claims the benefit, under 35 U.S.C. § 119(e), of U.S. Provisional Application No. 63/354,049, filed Jun. 21, 2022, the entire contents of which are incorporated by reference herein in their entirety.

### STATEMENT REGARDING ELECTRONIC FILING OF A SEQUENCE LISTING

A Sequence Listing in XML format, entitled 5470-932WO_ST26.xml, 397,082 bytes in size, generated on Jun. 21, 2023 and filed herewith, is hereby incorporated by reference in its entirety for its disclosures.

### FIELD OF THE INVENTION

The invention relates to the inhibition of expression of interferon regulatory factor-4 (IRF4) using RNA interference, chemically-modified oligonucleotides, and/or chimeric siRNA multivalent combinations. The invention further relates to methods of treating IRF4 related conditions such as multiple myeloma.

### BACKGROUND OF THE INVENTION

Multiple myeloma (MM) is a cancer of plasma cells that causes a host of problems including kidney failure, bony fractures, low blood counts and infections. Its annual incidence in the United States is roughly 30,000 new cases, with more than 10,000 deaths (Surveillance, Epidemiology, and End Results (SEER) Program; SEER*Stat Database: Populations—Total U.S. (1969-2020) <Katrina/Rita Adjustment>-Linked To County Attributes—Total U.S., 1969-2020 Counties, National Cancer Institute, DCCPS, Surveillance Research Program, released January 2022 In.) MM is treated primarily with systemic therapies; currently FDA-approved therapeutic classes/approaches include proteasome inhibitors, immunomodulatory agents, monoclonal antibodies, stem cell transplantation, and most recently, chimeric antigen receptor T-cells (CAR-T), among others. Despite that panoply of options, MM remains incurable and eventually becomes refractory to therapy. Patients generally progress over years through many lines of treatment, with the MM becoming progressively refractory to available agents with each new line of treatment. With that refractoriness MM becomes more difficult to control and causes more organ damage. Combined with cumulative therapy-related toxicity, almost all patients with MM eventually reach a point at which further systemic therapy is futile, with a slim likelihood of any treatment offering clinically meaningful disease control and high risk of significant toxicity. Advances in MM therapy have pushed that time point back in most patients-prognosis is better now than ever before-yet the fact that almost all patients with MM eventually succumb to MM and not other causes highlights the unmet need for additional effective agents that can control and even someday cure MM (Binder et al., Mortality trends in multiple myeloma after the introduction of novel therapies in the United States. Leukemia. 2022;36(3): 801-808)

Interferon regulatory factor-4 (IRF4) is a transcription factor that is critical for plasma cell regulation, where it impacts differentiation, growth, immunoglobulin class switching, metabolism, and immune activity, among other domains. IRF4 is often overexpressed in plasma cells that have become malignant (i.e., evolved into MM), and preclinical models have demonstrated that suppressing IRF4 reduces the viability of MM cells in vitro (Agnarelli et al., IRF4 in multiple myeloma-Biology, disease and therapeutic target. Leuk Res. 2018;72:52-58.). Despite that promise, until now IRF4 has not been thoroughly evaluated as a therapeutic target.

The present invention overcomes the deficiencies in the art by providing compositions and methods using RNA interference for specific inhibition of IRF4 and through the combination of dual IRF4 and c-Myc silencing.

### SUMMARY OF THE INVENTION

The present invention is based on the identification of RNA molecules that inhibit expression of IRF4 sequences. Accordingly, one aspect of the invention relates to a double stranded RNA molecule comprising an antisense strand and a sense strand, wherein the nucleotide sequence of the antisense strand is complementary to a region of the nucleotide sequence of a human IRF4 gene, the region consisting essentially of about 18 to about 25 consecutive nucleotides; wherein the double stranded RNA molecule inhibits expression of a human IRF4 gene. Recent work has revealed that IRF4 and c-Myc form a positive regulatory loop, suggesting that the ability to co-silence both targets may have additive and/or synergistic anti-cancer effects. In one embodiment, multiple siRNAs are used to inhibit both IRF4 and c-Mye genes simultaneously.

Another aspect of the invention relates to a composition, e.g., a pharmaceutical composition, comprising one or more of the RNA molecules of the invention.

A further aspect of the invention relates to a method of inhibiting expression of a human IRF4 gene, the method comprising contacting the cell with the RNA molecule of the invention, thereby inhibiting expression of the human IRF4 gene in the cell.

An additional aspect of the invention relates to a method of treating cancer in a subject in need thereof, wherein the cancer expresses or over-expresses a human IRF4 gene, the method comprising delivering to the subject the RNA molecule of the invention, thereby treating cancer in the subject.

Another aspect of the invention relates to the use of the RNA molecules of the invention to inhibit expression of a human IRF4 gene in a cell and to treat cancer in a subject in need thereof, wherein the cancer comprises over-expression of a human c-Myc gene.

Another aspect of the invention relates to a siRNA molecule targeted to a naturally-occurring human IRF4 mRNA, wherein the siRNA molecule comprises at least one chemical modification, and wherein the siRNA molecule comprises one of the following pairs of sequences:
sense strand of SEQ ID NO:1 and an antisense strand of SEQ ID NO:2;
sense strand of SEQ ID NO:3 and an antisense strand of SEQ ID NO:4;
sense strand of SEQ ID NO:5 and an antisense strand of SEQ ID NO:6;
sense strand of SEQ ID NO:7 and an antisense strand of SEQ ID NO:8;
sense strand of SEQ ID NO:9 and an antisense strand of SEQ ID NO:10;
sense strand of SEQ ID NO:11 and an antisense strand of SEQ ID NO:12;
sense strand of SEQ ID NO:13 and an antisense strand of SEQ ID NO:14;
sense strand of SEQ ID NO:15 and an antisense strand of SEQ ID NO:16;
sense strand of SEQ ID NO:17 and an antisense strand of SEQ ID NO:18;
sense strand of SEQ ID NO:19 and an antisense strand of SEQ ID NO:20;
sense strand of SEQ ID NO:21 and an antisense strand of SEQ ID NO:22;
sense strand of SEQ ID NO:23 and an antisense strand of SEQ ID NO:24;
   or a sequence at least 90% identical thereto.

Another aspect of the invention relates to a composition, e.g., a pharmaceutical composition, comprising one or more of the siRNA molecules of the invention.

A further aspect of the invention relates to a method of inhibiting expression of a human IRF4 gene in a cell, the method comprising contacting the cell with one or more of the siRNAs molecules of the invention, thereby inhibiting expression of the human IRF4 gene in the cell.

An additional aspect of the invention relates to a method of treating cancer in a subject in need thereof, wherein the cancer expresses or over-expresses a human IRF4 gene, the method comprising delivering to the subject the siRNA molecules of the invention, thereby treating cancer in the subject.

Another aspect of the invention relates to the use of the siRNA molecules of the invention to inhibit expression of a human IRF4 gene in a cell and to treat cancer in a subject in need thereof, wherein the cancer expresses or over-expresses a human IRF4 gene.

Another aspect of the invention relates to double stranded RNA molecules, a first RNA molecule comprising an antisense strand and a sense strand targeted to a human IRF4 gene and a second RNA molecule comprising an antisense strand and a sense strand targeted to a human c-Myc gene, wherein the nucleotide sequence of the first RNA molecule has an antisense strand that is complementary to a region of the nucleotide sequence of a human IRF4 gene, the region consisting essentially of about 18 to about 25 consecutive nucleotides; wherein the double stranded RNA molecule inhibits expression of a human IRF4 gene, and wherein the nucleotide sequence of the second RNA molecule has an antisense strand that is complementary to a region of the nucleotide sequence of a human c-Myc gene, the region consisting essentially of about 18 to about 25 consecutive nucleotides; wherein the double stranded RNA molecule inhibits expression of a human c-Myc gene.

Another aspect of the invention relates to a pair of siRNA molecules, comprsing a first siRNA molecule targeted to a naturally-occurring human IRF4 mRNA, wherein the siRNA molecule comprises at least one chemical modification, and wherein the siRNA molecule comprises one of the following pairs of sequences:
sense strand of SEQ ID NO:1 and an antisense strand of SEQ ID NO:2;
sense strand of SEQ ID NO:3 and an antisense strand of SEQ ID NO:4;
sense strand of SEQ ID NO:5 and an antisense strand of SEQ ID NO:6;
sense strand of SEQ ID NO:7 and an antisense strand of SEQ ID NO:8;
sense strand of SEQ ID NO:9 and an antisense strand of SEQ ID NO:10;
sense strand of SEQ ID NO:11 and an antisense strand of SEQ ID NO:12;
sense strand of SEQ ID NO:13 and an antisense strand of SEQ ID NO:14;
sense strand of SEQ ID NO:15 and an antisense strand of SEQ ID NO:16;
sense strand of SEQ ID NO:17 and an antisense strand of SEQ ID NO:18;
sense strand of SEQ ID NO:19 and an antisense strand of SEQ ID NO:20;
sense strand of SEQ ID NO:21 and an antisense strand of SEQ ID NO:22;
sense strand of SEQ ID NO:23 and an antisense strand of SEQ ID NO:24;
   or a sequence at least 90% identical thereto; and
   a second siRNA molecule targeted to a a naturally-occuring human c-Myc sequence, wherein the siRNA molecule comprises at least one chemical modification. In some emboidments, the seocnd siRNA molecule comprises one of the following pairs of sequences:
sense strand of SEQ ID NO:48 and an antisense strand of SEQ ID NO:49;
sense strand of SEQ ID NO:50 and an antisense strand of SEQ ID NO:51;
sense strand of SEQ ID NO:52 and an antisense strand of SEQ ID NO:53;
sense strand of SEQ ID NO:54 and an antisense strand of SEQ ID NO:55;
sense strand of SEQ ID NO:56 and an antisense strand of SEQ ID NO:57;
sense strand of SEQ ID NO:58 and an antisense strand of SEQ ID NO:59;
sense strand of SEQ ID NO:60 and an antisense strand of SEQ ID NO:61;
sense strand of SEQ ID NO:62 and an antisense strand of SEQ ID NO:63;
sense strand of SEQ ID NO:64 and an antisense strand of SEQ ID NO:65;
sense strand of SEQ ID NO:66 and an antisense strand of SEQ ID NO:67;
sense strand of SEQ ID NO:68 and an antisense strand of SEQ ID NO:69;
sense strand of SEQ ID NO:70 and an antisense strand of SEQ ID NO:71;
sense strand of SEQ ID NO:72 and an antisense strand of SEQ ID NO:73;
sense strand of SEQ ID NO:74 and an antisense strand of SEQ ID NO:75;
sense strand of SEQ ID NO:76 and an antisense strand of SEQ ID NO:77; or
sense strand of SEQ ID NO:78 and an antisense strand of SEQ ID NO:79;
   or a sequence at least 90% identical thereto.

Another aspect of the invention relates to a chimeric multivalent siRNA molecule targeted to one or more genes. In one embodiment, the chimeric siRNA molecule comprisies a first siRNA molecule targeted to a naturally-occurring human IRF4 mRNA, wherein the siRNA molecule comprises at least one chemical modification, and a second siRNA molecule targeted to a a naturally-occurring human c-Myc mRNA, wherein the siRNA molecule comprises at least one chemical modification, wherein the first siRNA molecule is attached to the second siRNA molecule by a linking region, forming the chimeric siRNA molecule.

Another aspect of the invention relates to a composition, e.g., a pharmaceutical composition, comprising one or more of the siRNA molecules of the invention.

A further aspect of the invention relates to a method of inhibiting expression of a human IRF4 gene and a human c-Myc gene in a cell, the method comprising contacting the cell with the siRNAs molecules of the invention, thereby inhibiting expression of the human IRF4 gene and the human c-Myc gene in the cell.

An additional aspect of the invention relates to a method of treating cancer in a subject in need thereof, wherein the cancer expresses or over-expresses a human IRF4 gene, the method comprising delivering to the subject siRNA molecules of the invention, thereby treating cancer in the subject.

Another aspect of the invention relates to the use of the siRNA molecules of the invention to inhibit expression of a human IRF4 gene in a cell and to treat cancer in a subject in need thereof, wherein the cancer expresses or over-expresses a human IRF4 gene.

These and other aspects of the invention are set forth in more detail in the description of the invention below.

### BRIEF DESCRIPTION OF THE DRAWINGS

FIG. 1 shows IRF4 is an “undruggable” protein.

FIG. 2 shows selected unmodified IRF4 siRNAs (SEQ ID NOS: 1-24).

FIG. 3 shows unmodified siRNAs decrease IRF4 levels in RPMI 8226 cells at 24 h. RPMI-8226 cells were treated with 20 nM unmodified siRNAs complexed with RNAiMax, as well as a negative control (snord90) and positive control (seq3) that targets KRAS. Afterwards, 24 hours post-transfection, cells were harvested for RT-qPCR analysis. mRNA levels were normalized against 18S or GAPDH.

FIG. 4 shows unmodified siRNAs decrease IRF4 and myc mRNA expression over time. RPMI-8226 cells were treated with 20 nM unmodified siRNAs complexed with RNAiMax, as well as a negative control (snord90) and positive control (seq3) that targets KRAS. Afterwards, 24, 48, and 72 hours post-transfection, cells were harvested for RT-qPCR analysis. mRNA levels were normalized against GAPDH.

FIG. 5 shows an immunoblot of unmodified IRF4 siRNAs in RPMI-8226 cells at 48 h. RPMI-8226 cells were treated with 20 nM unmodified siRNAs complexed with RNAiMax, as well as a negative control (snord90) and positive control (seq3). Afterwards, 48 hours post-transfection, cells were harvested for immunoblotting. Proteins of interest were quantified using ImageLab and normalized to a loading control, vinculin.

FIG. 6 shows IRF4 siRNAs selected for Hi2F nucleotide modification.

FIG. 7 shows Hi2F siRNAs decrease IRF4 and c-Myc levels in RPMI-8229 cells over time. RPMI-8226 cells were treated with 20 nM Hi2F modified siRNAs complexed with RNAiMax, as well as a negative control (snord90) and positive control (seq3) that targets KRAS. Afterwards, 24 and 48 hours post-transfection, cells were harvested for RT-qPCR analysis.

FIG. 8 shows IRF4 or Myc Hi2F siRNAs decrease mRNA levels in RPMI-8226 cells. RPMI-8226 cells were treated with 10 and 20 nM Hi2F modified siRNAs complexed with RNAiMax, as well as a negative control (2OMe). Afterwards, 24 and 72 hours post-transfection, cells were harvested for RT-qPCR analysis.

FIG. 9 shows key data of the IRF4 and Myc Hi2F siRNA experiment (FIG. 8).

FIG. 10 shows Hi2F siRNAs decrease IRF4 and c-Myc mRNA levels over time in KMS-11 cells. KMS-11 cells were treated with 20 nM Hi2F modified siRNAs complexed with RNAiMax, as well as a negative control (2OMe) and positive control (seq3) that targets KRAS. Afterwards, 24 and 48 hours post-transfection, cells were harvested for RT-qPCR analysis.

FIG. 11 shows Hi2F siRNAs decrease IRF4 and c-Myc protein expression in RPMI-8226 cells over time. RPMI-8226 cells were treated with 20 nM Hi2F siRNAs complexed with RNAiMax, as well as a negative control (snord90) and positive control (seq3). Afterwards, 48 and 72 hours post-transfection, cells were harvested for immunoblotting. Proteins of interest were quantified using ImageLab and normalized to a loading control, vinculin.

FIG. 12 shows Hi2F siRNAs targeting IRF4 decrease RPMI-8226 cell viability. RPMI-8226 cells were treated with varying doses of Hi2F modified siRNAs complexed with RNAiMax in 96-well plate format for 5 days, along with a negative control (2OMe). On the 5th day post-transfection, Cell Titer Glo 2.0 was added and luminescence was determined.

FIG. 13 shows Hi2F siRNAs targeting IRF4 or c-Myc decrease RPMI-8226 cell viability. RPMI-8226 cells were treated with varying doses of Hi2F modified siRNAs complexed with RNAiMax in 96-well plate format for 5 days, along with a negative control (2OMe). On the 5th day post-transfection, Cell Titer Glo 2.0 was added and luminescence was determined.

FIG. 14 shows Hi2F siRNAs targeting IRF4 decrease KMS-11 cell viability. KMS-11 cells were treated with varying doses of Hi2F modified siRNAs complexed with RNAiMax in 96-well plate format for 5 days, along with a negative control (2OMe). On the 5th day post-transfection, Cell Titer Glo 2.0 was added and luminescence was determined.

FIG. 15 shows IRF4 siRNAs selected for HiOMe modification.

FIG. 16 shows Hi2F versus HiOMe modifications have minimal effect on efficacy of siRs. RPMI-8226 cells were treated with 10 or 20 nM Hi2F and HiOMe modified siRNAs complexed with RNAiMax, as well as a negative control (2OMe) and positive control (seq3) that targets KRAS. Afterwards, 24 and 72 hours post-transfection, cells were harvested for RT-qPCR analysis.

FIG. 17 shows Hi2F versus HiOMe modifications have minimal effect on efficacy of siRNAs. RPMI-8226 cells were treated with 20 nM Hi2F and HiOMe siRNAs complexed with RNAiMax, as well as a negative control (2OMe) and positive control (seq3). Afterwards, 48 and 72 hours post-transfection, cells were harvested for immunoblotting. Proteins of interest were quantified using ImageLab and normalized to a loading control, vinculin.

FIG. 18 shows quantitation of IRF4 siR knockdown of protein expression.

FIG. 19 shows Hi2F versus HiOMe modifications have minimal effect on efficacy of siRs. RPMI-8226 cells were treated with varying doses of Hi2F or HiOme modified siRNAs complexed with RNAiMax in 96-well plate format for 5 days, along with a negative control (2OMe). On the 5th day post-transfection, Cell Titer Glo 2.0 was added and luminescence was determined.

FIG. 20 shows Hi2F and HiOMe versions of siR10 both decrease RPMI-8226 cell counts. RPMI-8226 cells were treated with 20 nM Hi2F or HiOme modified siR10 complexed with RNAiMax for 6 days, along with a negative control (2OMe). Total Cell Area of brightfield images were quantified using ImageJ.

FIG. 21 shows IRF4 siR10-HiOMe creates cellular toxicity in RPMI-8226 cells. RPMI-8226 cells were treated with varying doses of HiOme modified siR10 and a negative control (2OMe) complexed with RNAiMax in 96-well plate format with CellTox reagent. Each day for six days, fluorescence of CellTox was read with a luminometer. On the 6th day, Cell Titer Glo 2.0 was added and luminescence was determined for a viability readout.

FIG. 22 shows IRF4 siR10-HiOMe creates cellular toxicity in RPMI-8226 cells. RPMI-8226 cells were treated with varying doses of HiOme modified siR10 and a negative control (2OMe) complexed with RNAiMax in 96-well plate format with CellTox reagent. Each day for six days, fluorescence of CellTox was read with a luminometer. On the 6th day, Cell Titer Glo 2.0 was added and luminescence was determined for a viability readout.

FIG. 23 shows siR 10 Hi2F and HiOMe siRNAs both increase cellular toxicity and decrease viability in spheroids of RPMI-8226 cells. RPMI-8226 cells were transfected with 20 nM Hi2F or HiOMe modified siRNAs with RNAiMax, along with negative control (2OMe). After 24 hours, 5,000 (5 k) or 10,000 (10 k) cells were re-plated in 96 well plates in the presence of CellTox reagent, and spheroids were formed by centrifugation. Cells were imaged every 5 hours for 6 days in an Incucyte. On the 6th day, Cell-Titer Glo 3D was added to ascertain cellular viability.

FIG. 24 shows siR 10-HiOMe inhibits agarose colony formation of myeloma cells. RPMI-8226 cells were treated with 20 nM HiOMe modified siR10 complexed with RNAiMax, as well as a negative control (2OMe). After 24 hrs, cells were replated in 0.3% agarose. After 16 days, cells were visualized with iodonitrotetrazolium chloride, and images were quantified with OrganoSeg.

FIG. 25 shows siR 10-HiOMe inhibits agarose colony formation of myeloma cells. RPMI-8226 cells were treated with 20 nM HiOMe modified siR10 complexed with RNAiMax, as well as a negative control (2OMe). After 24 hrs, cells were replated in 0.3% agarose. After 16 days, cells were visualized with iodonitrotetrazolium chloride, and images were quantified with OrganoSeg.

FIG. 26 shows Myc2-siR10 chimera has a significant effect on myeloma cell viability. RPMI-8226 cells were treated with varying doses of HiOMe siRNAs complexed with RNAiMax in 96-well plate format for 5 days, with 2OMe as the negative control. On the 5th day post-transfection, Cell Titer Glo 2.0 was added and luminescence was determined.

FIG. 27 shows HiOMe modified IRF4 and myc-IRF4 chimera siRNAs produce cellular toxicity in RPMI-8226 cells. RPMI-8226 cells were treated with varying doses of HiOme modified siR10, myc2 or chimera siRNAs complexed with RNAiMax in 96-well plate format with CellTox reagent, along with a negative control (2OMe). Each day for six days, fluorescence of CellTox was read with a luminometer.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Green et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

### Definitions

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide sequence of this invention, means a polynucleotide that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to inhibit expression of a target mRNA of at least about 50% or more as compared to the expression level of a polynucleotide consisting of the recited sequence.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of cancer, reduction in tumor burden, prevention of further tumor growth, prevention of metastasis, or increase in survival time). Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

“Prevent” or “preventing” or “prevention” refer to prevention or delay of the onset of the disorder and/or a decrease in the severity of the disorder in a subject relative to the severity that would develop in the absence of the methods of the invention. The prevention can be complete, e.g., the total absence of cancer in a subject. The prevention can also be partial, such that the occurrence or severity of cancer in a subject is less than that which would have occurred without the present invention.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and/or adenovirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Felgner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Felgner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931).

It is also possible to introduce a vector in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

As used herein, the terms “protein” and “polypeptide” are used interchangeably and encompass both peptides and proteins, unless indicated otherwise.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, etc.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

As used herein, the term “over-expression” or “over-expressing” refers to increased levels of a polypeptide being produced and/or increased time of expression (e.g., constitutively expressed) compared to a wild-type cell.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, “complementary” polynucleotides are those that are capable of base pairing according to the standard Watson-Crick complementarity rules. Specifically, purines will base pair with pyrimidines to form a combination of guanine paired with cytosine (G: C) and adenine paired with either thymine (A: T) in the case of DNA, or adenine paired with uracil (A: U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least about 50%, 60%, 70%, 80% or 90% of their nucleotides. In some embodiments, the two nucleic acid sequences can be complementary at least at 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

As used herein, the terms “contacting,” “introducing” and “administering” are used interchangeably, and refer to a process by which dsRNA of the present invention or a nucleic acid molecule encoding a dsRNA of this invention is delivered to a cell, in order to inhibit or alter or modify expression of a target gene. The dsRNA may be administered in a number of ways, including, but not limited to, direct introduction into a cell (i.e., intracellularly) and/or extracellular introduction into a cavity, interstitial space, or into the circulation of the organism.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

“Transient transformation” or “transient transfection” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stable transfection” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and mitochondrial genome, and therefore includes integration of the nucleic acid into, for example, the mitochondrial genome. Stable transformation or stable transfection as used herein can also refer to a transgene that is maintained extrachromasomally, for example, as a minichromosome.

Transient transformation or transient tranfection may be detected by, for example, an enzyme-linked immunosorbent assay (ELISA) or Western blot, which can detect the presence of a peptide or polypeptide encoded by one or more transgene introduced into an organism. Stable transformation or stable transfection of a cell can be detected by, for example, a Southern blot hybridization assay of genomic DNA of the cell with nucleic acid sequences which specifically hybridize with a nucleotide sequence of a transgene introduced into an organism. Stable transformation or stable transfection of a cell can be detected by, for example, a Northern blot hybridization assay of RNA of the cell with nucleic acid sequences which specifically hybridize with a nucleotide sequence of a transgene introduced into an organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Embodiments of the invention are directed to expression cassettes designed to express the nucleic acids of the present invention. As used herein, “expression cassette” means a nucleic acid molecule having at least a control sequence operably linked to a nucleotide sequence of interest. In this manner, for example, promoters in operable interaction with the nucleotide sequences for the siRNAs of the invention are provided in expression cassettes for expression in an organism or cell.

As used herein, the term “promoter” refers to a region of a nucleotide sequence that incorporates the necessary signals for the efficient expression of a coding sequence. This may include sequences to which an RNA polymerase binds, but is not limited to such sequences and can include regions to which other regulatory proteins bind together with regions involved in the control of protein translation and can also include coding sequences.

Furthermore, a “promoter” of this invention is a promoter capable of initiating transcription in a cell of an organism. Such promoters include those that drive expression of a nucleotide sequence constitutively, those that drive expression when induced, and those that drive expression in a tissue-or developmentally-specific manner, as these various types of promoters are known in the art.

For purposes of the invention, the regulatory regions (i.e., promoters, transcriptional regulatory regions, and translational termination regions) can be native/analogous to the organism or cell and/or the regulatory regions can be native/analogous to the other regulatory regions. Alternatively, the regulatory regions may be heterologous to the organism or cell and/or to each other (i.e., the regulatory regions). Thus, for example, a promoter can be heterologous when it is operably linked to a polynucleotide from a species different from the species from which the polynucleotide was derived. Alternatively, a promoter can also be heterologous to a selected nucleotide sequence if the promoter is from the same/analogous species from which the polynucleotide is derived, but one or both (i.e., promoter and polynucleotide) are substantially modified from their original form and/or genomic locus, or the promoter is not the native promoter for the operably linked polynucleotide.

The choice of promoters to be used depends upon several factors, including, but not limited to, cell-or tissue-specific expression, desired expression level, efficiency, inducibility and selectability. For example, where expression in a specific tissue or organ is desired, a tissue-specific promoter can be used. In contrast, where expression in response to a stimulus is desired, an inducible promoter can be used. Where continuous expression is desired throughout the cells of an organism, a constitutive promoter can be used. It is a routine matter for one of skill in the art to modulate the expression of a nucleotide sequence by appropriately selecting and positioning promoters and other regulatory regions relative to that sequence.

In addition to the promoters described above, the expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences and polyadenylation signal sequences.

The expression cassette also can optionally include a transcriptional and/or translational termination region (i.e., termination region) that is functional in the organism. A variety of transcriptional terminators are available for use in expression cassettes and are responsible for the termination of transcription beyond the transgene and correct mRNA polyadenylation. The termination region may be native to the transcriptional initiation region, may be native to the operably linked nucleotide sequence of interest, may be native to the host, or may be derived from another source (i.e., foreign or heterologous to the promoter, the nucleotide sequence of interest, the host, or any combination thereof).

A signal sequence can be operably linked to nucleic acids of the present invention to direct the nucleotide sequence into a cellular compartment. In this manner, the expression cassette will comprise a nucleotide sequence encoding the siRNA operably linked to a nucleic acid sequence for the signal sequence. The signal sequence may be operably linked at the N- or C-terminus of the siRNA.

Regardless of the type of regulatory sequence(s) used, they can be operably linked to the nucleotide sequence of the siRNA. As used herein, “operably linked” means that elements of a nucleic acid construct such as an expression cassette are configured so as to perform their usual function. Thus, regulatory or control sequences (e.g., promoters) operably linked to a nucleotide sequence of interest are capable of effecting expression of the nucleotide sequence of interest. The control sequences need not be contiguous with the nucleotide sequence of interest, so long as they function to direct the expression thereof. Thus, for example, intervening untranslated, yet transcribed, sequences can be present between a promoter and a coding sequence, and the promoter sequence can still be considered “operably linked” to the coding sequence. A nucleotide sequence of the present invention (i.e., a siRNA) can be operably linked to a regulatory sequence, thereby allowing its expression in a cell and/or subject.

The expression cassette also can include a nucleotide sequence for a selectable marker, which can be used to select a transformed organism or cell. As used herein, “selectable marker” means a nucleic acid that when expressed imparts a distinct phenotype to the organism or cell expressing the marker and thus allows such transformed organisms or cells to be distinguished from those that do not have the marker. Such a nucleic acid may encode either a selectable or screenable marker, depending on whether the marker confers a trait that can be selected for by chemical means, such as by using a selective agent (e.g., an antibiotic or the like), or on whether the marker is simply a trait that one can identify through observation or testing, such as by screening (. Of course, many examples of suitable selectable markers are known in the art and can be used in the expression cassettes described herein.

In some embodiments of the present invention, the expression cassette can comprise an expression control sequence operatively linked to a nucleotide sequence that is a template for one or both strands of the dsRNA. In further embodiments, a promoter can flank either end of the template nucleotide sequence, wherein the promoters drive expression of each individual DNA strand, thereby generating two complementary (or substantially complementary) RNAs that hybridize and form the dsRNA. In alternative embodiments, the nucleotide sequence is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 basepairs, and wherein after transcription, the RNA transcript folds on itself to form a short hairpin RNA (shRNA) molecule.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48: 1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, “RNAi” or “RNA interference” refers to the process of sequence-specific post-transcriptional gene silencing, mediated by double-stranded RNA (dsRNA). As used herein, “dsRNA” refers to RNA that is partially or completely double stranded. Double stranded RNA is also referred to as small interfering RNA (siRNA), small interfering nucleic acid (siNA), microRNA (miRNA), and the like. In the RNAi process, dsRNA comprising a first (antisense) strand that is complementary to a portion of a target gene and a second (sense) strand that is fully or partially complementary to the first antisense strand is introduced into an organism. After introduction into the organism, the target gene-specific dsRNA is processed into relatively small fragments (siRNAs) and can subsequently become distributed throughout the organism, leading to a phenotype that, over the period of a generation, may come to closely resemble the phenotype arising from a complete or partial deletion of the target gene.

MicroRNAs (miRNAs) are non-protein coding RNAs, generally of between about 18 to about 25 nucleotides in length. These miRNAs direct cleavage in trans of target transcripts, negatively regulating the expression of genes involved in various regulation and development pathways (Bartel, Cell, 116:281-297 (2004); Zhang et al. Dev. Biol. 289:3-16(2006)). As such, miRNAs have been shown to be involved in different aspects of growth and development as well as in signal transduction and protein degradation. Since the first miRNAs were discovered in plants (Reinhart et al. Genes Dev. 16:1616-1626 (2002), Park et al. Curr. Biol. 12:1484-1495 (2002)) many hundreds have been identified. Many microRNA genes (MIR genes) have been identified and made publicly available in a database (miRBase; microrna.sanger.ac.uk/sequences). miRNAs are also described in U.S. Patent Publications 2005/0120415 and 2005/144669A1, the entire contents of which are incorporated by reference herein.

Genes encoding miRNAs yield primary miRNAs (termed a “pri-miRNA”) of 70 to 300 bp in length that can form imperfect stem-loop structures. A single pri-miRNA may contain from one to several miRNA precursors. In animals, pri-miRNAs are processed in the nucleus into shorter hairpin RNAs of about 65 nt (pre-miRNAs) by the RNaseIII enzyme Drosha and its cofactor DGCR8/Pasha. The pre-miRNA is then exported to the cytoplasm, where it is further processed by another RNaseIII enzyme, Dicer, releasing a miRNA/miRNA* duplex of about 22 nt in size. Many reviews on microRNA biogenesis and function are available, for example, see, Bartel Cell 116:281-297 (2004), Murchison et al. Curr. Opin. Cell Biol. 16:223-229 (2004), Dugas et al. Curr. Opin. Plant Biol. 7:512-520(2004) and Kim Nature Rev. Mol. Cell Biol. 6:376-385 (2005).

### RNA Molecules

The invention presents an alternative therapeutic approach by targeting IRF4 at the transcriptional level. The invention consists of dsRNA molecules (e.g., short interfering RNAs (siRNAs)) that can complementarily bind to IRF4 messenger RNA and inhibit transcription of the oncogene, inducing gene knockdown and subsequent apoptosis in cancer cells. Traditionally, siRNAs have faced clinical limitations due to their lack of tissue specificity, rapid degradation within the body, and immune activation. However, the synthetic siRNAs herein contain novel chemical modifications that confer drug-like properties, which will protect them from in vivo degradation and displace the need for a nanocarrier or other delivery system.

Accordingly, one aspect of the invention relates to a double stranded RNA molecule comprising an antisense strand and a sense strand, wherein the nucleotide sequence of the antisense strand is complementary to a region of the nucleotide sequence of a human IRF4gene, the region comprsing, consisting essentially of, or ocnsisting of about 18 to about 25 consecutive nucleotides; wherein the double stranded RNA molecule inhibits expression of a human IRF4 gene. The RNA molecules provide decreased expression of IRF4 in a cell as compared to cells without the RNA molecules (e.g., a control cell or nontransformed cell). In some embodiments, expression of IRF4 is inhibited by at least about 50%, e.g., at least about 50%, 60%, 70%, 80%, 90%, 95%, or more.

The double stranded RNA molecule can comprise, consist essentially of, or consist of about 18 to about 25 nucleotides (e.g., 18, 19, 20, 21, 22, 23, 24, or 25 or any range therein). Additional nucleotides can be added at the 3′ end, the 5′ end or both the 3′ and 5′ ends to facilitate manipulation of the RNA molecule but that do not materially affect the basic characteristics or function of the double stranded RNA molecule in RNA interference (RNAi). Additionally, one or two nucleotides can be deleted from one or both ends of any of the sequences disclosed herein that do not materially affect the basic characteristics or function of the double stranded RNA molecule in RNAi. The term “materially affect” as used herein refers to a change in the ability to inhibit expression of the protein encoded by the mRNA by no more than about 50%, e.g., no more than about 50%, 45%, 40%, 35%, 30%, 25%, 20%, 15%, 10%, or less. Such additional nucleotides can be nucleotides that extend the complementarity of the antisense strand along the target sequence and/or such nucleotides can be nucleotides that facilitate manipulation of the RNA molecule or a nucleic acid molecule encoding the RNA molecule, as would be known to one of skill in the art. For example, a TT overhang at the 3′ end may be present, which is used to stabilize the siRNA duplex and does not affect the specificity of the siRNA.

The dsRNA of the invention may optionally comprise a single stranded overhang at either or both ends. The double-stranded structure may be formed by a single self-complementary RNA strand (i.e., forming a hairpin loop) or two complementary RNA strands. RNA duplex formation may be initiated either inside or outside the cell. When the dsRNA of the invention forms a hairpin loop, it may optionally comprise an intron and/or a nucleotide spacer, which is a stretch of nucleotides between the complementary RNA strands, to stabilize the hairpin sequence in cells. The RNA may be introduced in an amount that allows delivery of at least one copy per cell. Higher doses of double-stranded material may yield more effective inhibition.

In particular embodiments, the present invention provides double stranded RNA containing a nucleotide sequence that is fully complementary to a region of the target gene for inhibition. However, it is to be understood that 100% complementarity between the antisense strand of the double stranded RNA molecule and the target sequence is not required to practice the present invention. Thus, sequence variations that might be expected due to genetic mutation, strain polymorphism, or evolutionary divergence can be tolerated. RNA sequences with insertions, deletions, and single point mutations relative to the target sequence may also be effective for inhibition.

In some embodiments, the nucleotide sequence of the sense and antisense strands comprises a nucleotide sequence that is at least about 80% identical to the nucleotide sequence of any of SEQ ID NOS: 1-24, e.g., at least about 80%, 85%, 90%, 91%, 92%, 93%, 94%, 95%, 96%, 97%, 98%, 99%, or more identical to the nucleotide sequence of any of SEQ ID NOS: 1-24. In some embodiments, the nucleotide sequence of the sense and antisense strands of the doubles tranded RNA comprises, consists essentially of, one of the following pairs of sequences.
sense strand of SEQ ID NO: 1 and an antisense strand of SEQ ID NO:2;
sense strand of SEQ ID NO:3 and an antisense strand of SEQ ID NO:4;
sense strand of SEQ ID NO:5 and an antisense strand of SEQ ID NO:6;
sense strand of SEQ ID NO:7 and an antisense strand of SEQ ID NO:8;
sense strand of SEQ ID NO:9 and an antisense strand of SEQ ID NO:10;
sense strand of SEQ ID NO:11 and an antisense strand of SEQ ID NO:12;
sense strand of SEQ ID NO:13 and an antisense strand of SEQ ID NO:14;
sense strand of SEQ ID NO:15 and an antisense strand of SEQ ID NO:16;
sense strand of SEQ ID NO:17 and an antisense strand of SEQ ID NO:18;
sense strand of SEQ ID NO:19 and an antisense strand of SEQ ID NO:20;
sense strand of SEQ ID NO:21 and an antisense strand of SEQ ID NO:22;
sense strand of SEQ ID NO:23 and an antisense strand of SEQ ID NO:24;
   or a sequence at least 80% identical thereto, e.g., at least about 80%, 85%, 90%, 91%, 92%, 93%, 94%, 95%, 96%, 97%, 98%, 99%, or more identical thereto.

In some embodiments, one or both of the sense strand and the antisense strand comprises a TT overhang or other dinucleotide overhang at the 3′ end.

In some embodiments of this invention, the sense strand of the double stranded RNA molecule can be fully complementary to the antisense strand or the sense strand can be substantially complementary or partially complementary to the antisense strand. By substantially or partially complementary is meant that the sense strand and the antisense strand can be mismatched at about 1, 2, 3, 4, 5, 6, 7, 8, 9, or 10 nucleotide pairings. Such mismatches can be introduced into the sense strand sequence, e.g., near the 3′ end, to enhance processing of the double stranded RNA molecule by Dicer, to duplicate a pattern of mismatches in a siRNA molecule inserted into a chimeric nucleic acid molecule or artificial microRNA precursor molecule of this invention, and the like, as would be known to one of skill in the art. Such modification will weaken the base pairing at one end of the duplex and generate strand asymmetry, therefore enhancing the chance of the antisense strand, instead of the sense strand, being processed and silencing the intended gene (Geng and Ding “Double-mismatched siRNAs enhance selective gene silencing of a mutant ALS-causing Allelel” Acta Pharmacol. Sin. 29:211-216 (2008); Schwarz et al. “Asymmetry in the assembly of the RNAi enzyme complex” Cell 115:199-208 (2003)).

The double stranded RNA molecule of the invention may be in the form of any type of RNA interference molecule known in the art. In some embodiments, the double stranded RNA molecule is a small interfering RNA (siRNA) molecule. In other embodiments, the double stranded RNA molecule is a short hairpin RNA (shRNA) molecule. In other embodiments, the double stranded RNA molecule is part of a microRNA precursor molecule.

### Chemically-Modified siRNAs

One aspect of the invention relates to a siRNA molecule targeted to a IRF4 mRNA, wherein the siRNA comprises as least one chemical modification. In some embodiments, the siRNA molecule is fully chemically modified. The term “fully chemically-modified” means that every nucleotide in the siRNA contains a chemical modification. In some embodiments, each nucleotide in the siRNA molecule is modified with a 2′-O-methyl group or a 2′-fluoro group.

In certain embodiments, at least 1, 2, 3, 4, 5, 6, 7, 8, 9, 10, 11, 12, 13, 14, 15, 16, 17, 18, or 19 of the nucleotide linkages in the siRNA are chemically modified. In some embodiments, the siRNA comprises at least one phosphorothioate linkage. In some embodiments, the siRNA comprises 1, 2, 3, 4, 5, 6, 7, 8, 9, 10, 11, 12, 13, 14, 15, 16, 17, 18, or 19 phosphorothioate linkages. In some embodiments, the siRNA comprises all phosphorothioate linkages.

In certain embodiments, the siRNA molecule comprising at least one chemical modification comprises a sense strand and an antisense strand, wherein the siRNA molecule comprises one of the following pairs of sequences:
sense strand of SEQ ID NO:25 and an antisense strand of SEQ ID NO:26;
sense strand of SEQ ID NO:27 and an antisense strand of SEQ ID NO:28;
sense strand of SEQ ID NO:29 and an antisense strand of SEQ ID NO:30;
sense strand of SEQ ID NO:31 and an antisense strand of SEQ ID NO:32;
sense strand of SEQ ID NO:33 and an antisense strand of SEQ ID NO:34;
sense strand of SEQ ID NO:35 and an antisense strand of SEQ ID NO:36;
sense strand of SEQ ID NO:37 and an antisense strand of SEQ ID NO:38;
sense strand of SEQ ID NO:39 and an antisense strand of SEQ ID NO:40;
sense strand of SEQ ID NO:41 and an antisense strand of SEQ ID NO:42; or
sense strand of SEQ ID NO:43 and an antisense strand of SEQ ID NO:44.

In some embodiments, the siRNA molecule can be combined in tandem (e.g., administered together either unlinked or covalently or non-covalently linked) with a second siRNA molecule, e.g., one targeted to human c-Myc mRNA. In some embodiments, the siRNA molecule targeted to human c-Myc mRNA is partially or fully chemically modified. In some embodiments, the c-Myc siRNA comprises a sense strand and an antisense strand, wherein the siRNA molecule comprises one of the pairs of sequences listed in Table 1 and Table 2 or a sequence at least 90% identical thereto, e.g., at least 90%, 91%, 92%, 93%, 94%, 95%, 96%, 97%, 98%, or 99% identical thereto.

TABLE 1
c-Myc siRNA sequences

| | S | Sense Strand | | |
|---|---|---|---|---|
| | | Sequence | | |
| | | (5′ to 3′) | | |
| | A | Antisense | | |
| | | Strand | | SEQ |
| Oligo | | Sequence | | ID |
| Name | | (5′ to 3′) | Overhang | NO |
| cMyc1 | S | GAGGAUAUCUG | [dT] | 48 |
| | | GAAGAAAU | [dT] | |
| | A | AUUUCUUCCAG | [dT] | 49 |
| | | AUAUCCUC | [dT] | |
| cMyc2 | S | GAGAACAGUUG | [dT] | 50 |
| | | AAACACAA | [dT] | |
| | A | UUGUGUUUCAA | [dT] | 51 |
| | | CUGUUCUC | [dT] | |
| cMyc3 | S | AACACAAACUU | [dT] | 52 |
| | | GAACAGCU | [dT] | |
| | A | AGCUGUUCAAG | [dT] | 53 |
| | | UUUGUGUU | [dT] | |
| cMyc4 | S | AAGAAGAUGAG | [dT] | 54 |
| | | GAAGAAAU | [dT] | |
| | A | AUUUCUUCCUC | [dT] | 55 |
| | | AUCUUCUU | [dT] | |
| cMyc5 | S | CACAGCCCACU | [dT] | 56 |
| | | GGUCCUCAAGA | [dT] | |
| | A | UCUUGAGGACC | [dT] | 57 |
| | | AGUGGGCUGUG | [dT] | |
| cMyc6 | S | AAGAGGCGAAC | [dT] | 58 |
| | | ACACAACGUC | [dT] | |
| | A | GACGUUGUGUG | [dT] | 59 |
| | | UUCGCCUCUU | [dT] | |
| cMyc7 | S | CAGAUCAGCAA | [dT] | 60 |
| | | CAACCG | [dT] | |
| | A | CGGUUGUUGCU | [dT] | 61 |
| | | GAUCUG | [dT] | |
| cMyc8 | S | GAGACCUUCAU | [dT] | 62 |
| | | CAAAAACAUCA | [dT] | |
| | | UCA | | |
| | A | UGAUGAUGUUU | [dT] | 63 |
| | | UUGAUGAAGGU | [dT] | |
| | | CUC | | |
| Positive | S | GAGCUAAAACG | [dT] | 64 |
| Control | | GAGCUUUU | [dT] | |
| | A | AAAAGCUCCGU | [dT] | 65 |
| | | UUUAGCUC | [dT] | |

TABLE 2
Fully modified c-Myc siRNA sequences

| | | | SEQ |
|---|---|---|---|
| | | Sequence | ID |
| Name | Strand | (5′-3′) | NO |
| cMYC | S | [2flG]*[mC]*[2flG] | 66 |
| SiRNA 1 | | [mA][2flG][mG] | |
| Hi2F | | [2flA][mU][2flA] | |
| (Mseq1) | | [2flU][2flC][mU] | |
| | | [2flG][mG][2flA] | |
| | | [mA][2flG][mA] | |
| | | [2flA][mA][2flU] | |
| | AS | [mA]*[2flU]*[mU] | 67 |
| | | [2flU][mC][2flU] | |
| | | [mU][2flC][mC] | |
| | | [2flA][mG][mA] | |
| | | [mU][2flA][mU] | |
| | | [2flC][mC][2flU] | |
| | | [mC][2flG][mC] | |
| | | *[mU]*[mG] | |
| cMYC | S | [2fl]*[mC]*[2flG][mA] | |
| SiRNA 2 | | [2flG][mA][2flA][mC] | |
| Hi2F | | [2flA][2flG][2flU] | |
| (Mseq2) | | [mU][2flG][mA][2flA] | 68 |
| | | [mA][2flC][mA][2flC] | |
| | | [mA][2flA] | |
| | AS | [mU]*[2flU]*[mG][2flU] | 69 |
| | | [mG][2flU][mU][2flU] | |
| | | [mC][2flA][mA][mC] | |
| | | [mU][2flG][mU][2flU] | |
| | | [mC][2flU][mC][2flG] | |
| | | [mU]*[mC]*[mG] | |
| cMYC | S | [2flA]*[mC]*[2flA][mA] | 70 |
| SiRNA 3 | | [2flG][mA][2flA][mG] | |
| Hi2F | | [2flA][2flU][2flG] | |
| (Mseq3) | | [mA][2flG][mG][2flA] | |
| | | [mA][2flG][mA][2flA] | |
| | | [mA][2flU] | |
| | AS | [mA]*[2flU]*[mU][2flU] | 71 |
| | | [mC][2flU][mU][2flC] | |
| | | [mC][2flU][mC][mA] | |
| | | [mU][2flC][mU][2flU] | |
| | | [mC][2flU][mU][2flG] | |
| | | [mU]*[mU]*[mC] | |
| cMYC | S | [2flA]*[mA]*[2flG][mA] | 72 |
| SiRNA 4 | | [2flG][mG][2flC][mG] | |
| Hi2F | | [2flA][2flA][2flC] | |
| (Mseq4) | | [mA][2flC][mA][2flC] | |
| | | [mA][2flA][mC][2flG] | |
| | | [mU][2flC] | |
| | AS | [mG]*[2flA]*[mC][2flG] | 73 |
| | | [mU][2flU][mG][2flU] | |
| | | [mG][2flU][mG][mU] | |
| | | [mU][2flC][mG][2flC] | |
| | | [mC][2flU][mC][2flU] | |
| | | [mU]*[mG]*[mA] | |
| cMYC | S | [2flG]*[mA]*[2flG][mA] | 74 |
| SiRNA 5 | | [2flG][mC][2flU][mA] | |
| Hi2F | | [2flA][2flA][2flA] | |
| (Mseq5) | | [mC][2flG][mG][2flA] | |
| | | [mG][2flC][mU][2flU] | |
| | | [mU][2flU] | |
| | AS | [mA]*[2flA]*[mA][2flA] | 75 |
| | | [mG][2flC][mU][2flC] | |
| | | [mC][2flG][mU][mU] | |
| | | [mU][2flU][mA][2flG] | |
| | | [mC][2flU][mC][2flU] | |
| | | [mC]*[mG]*[mU] | |
| KRAS | S | [mG]*[mU]*[mC][mU][mC] | 76 |
| Seq2- | | [mU][2flU][mG][2flG] | |
| FM | | [2flA][2flU][mA][mU] | |
| | | [mU][mC][mU][mC][mG] | |
| | | [mA] | |
| | AS | [mU]*[2flC]*[mG][mA] | 77 |
| | | [mG][2flA][mA][mU] | |
| | | [mA][mU][mC][mC][mA] | |
| | | [2flA][mG][2flA][mG] | |
| | | [mA][mC]*[mA]*[mG] | |
| KRAS | S | [mC]*[mA]*[mG][mC][mU] | 78 |
| Seq3- | | [mA][2flA][mU][2flU] | |
| FM | | [2flC][2flA][mG][mA] | |
| | | [mA][mU][mC][mA][mU] | |
| | | [mU] | |
| | AS | [mA]*[2flA]*[mU][mG] | 79 |
| | | [mA][2flU][mU][mC] | |
| | | [mU][mG][mA][mA][mU] | |
| | | [2flU][mA][2flG][mC] | |
| | | [mU][mG]*[mU]*[mA] | |

S - sense strand
AS - antisense strand
m - 2′-O-methyl on sugar moieties
2fl - 2′-fluoro on sugar moieties
* - phosphorothioate in between nucleotides

In certain embodiments, at least 1, 2, 3, 4, 5, 6, 7, 8, 9, 10, 11, 12, 13, 14, 15, 16, 17, 18, or 19 of the nucleotide linkages in the c-Myc-targeted siRNA are chemically modified. In some embodiments, the c-Myc-targeted siRNA comprises at least one phosphorothioate linkage. In some embodiments, the c-Myc-targeted siRNA comprises 1, 2, 3, 4, 5, 6, 7, 8, 9, 10, 11, 12, 13, 14, 15, 16, 17, 18, or 19 phosphorothioate linkages. In some embodiments, the-targeted siRNA comprises all phosphorothioate linkages.

The double stranded RNA molecule or chemically-modified siRNA molecule may be constructed using chemical synthesis and enzymatic ligation reactions by procedures known in the art. For example, a double stranded RNA or chemically-modified siRNA molecule may be chemically synthesized using naturally occurring nucleotides or various modified nucleotides designed to increase the biological stability of the molecules or to increase the physical stability of the duplex formed between the double stranded RNA or chemically-modified siRNA molecule and target nucleotide sequences, e.g., phosphorothioate derivatives and acridine substituted nucleotides can be used. Examples of modified nucleotides which can be used to generate the double stranded RNA or chemically-modified siRNA molecule include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the double stranded RNA can be produced using an expression vector into which a nucleic acid encoding the double stranded RNA has been cloned.

The double stranded RNA or chemically-modified siRNA molecule can further include nucleotide sequences wherein at least one, or all, of the internucleotide bridging phosphate residues are modified phosphates, such as methyl phosphonates, methyl phosphonothioates, phosphoromorpholidates, phosphoropiperazidates and phosphoramidates. For example, every one or every other one of the internucleotide bridging phosphate residues can be modified as described. In another non-limiting example, the double stranded RNA or chemically-modified siRNA molecule is a nucleotide sequence in which at least one, or all, of the nucleotides contain a 2′ lower alkyl moiety (e.g., C₁-C_{4,} linear or branched, saturated or unsaturated alkyl, such as methyl, ethyl, ethenyl, propyl, 1-propenyl, 2-propenyl, and isopropyl). In another example, one or more of the nucleotides may be a 2′-fluoro nucleotide, a 2-O-methyl nucleotide, or a locked nucleic acid nucleotide. For example, every one or every other one of the nucleotides can be modified as described. See also, Furdon et al., Nucleic Acids Res. 17:9193 (1989); Agrawal et al., Proc. Natl. Acad. Sci. USA 87:1401 (1990); Baker et al., Nucleic Acids Res. 18:3537 (1990); Sproat et al., Nucleic Acids Res. 17:3373 (1989); Walder and Walder, Proc. Natl. Acad. Sei. USA 85:5011 (1988); incorporated by reference herein in their entireties for their teaching of methods of making polynucleotide molecules, including those containing modified nucleotide bases).

The invention further relates to a RNA molecule that is a multivalent chimeric double stranded RNA or chemically-modified siRNA molecule. In some embodiments, the multivalent chimeric siRNA molecule targets multiple genes, with each siRNA connected by metabolically-vulnerable linkers, e.g., 2, 3, 4, 5 or more genes. In some embodiments, the multivalent chimera comprises at least one siRNA targeted to IRF4 and at least one siRNA targeted to any other gene. In some embodiments, the multivalent chimera can be used to deliver two or more siRNAs targeting the same gene, e.g., 2, 3, 4, 5, 6, 7, 8, 9, 10, or more. The chimera can be formed of two or more double stranded RNA or chemically-modified siRNA molecules connected by a metabolically-vulnerable nucleotide linker (e.g., phosphodiester thymines, phosphodiester adenines, a phosphodiester TCA linker, etc.), e.g., comprising 2 or more nucleotides, e.g., 2, 3, 4, 5, 6, 7, 8, 9, or 10 nucleotides or more, e.g., 2-6 or 3-5 nucleotides.

In some embodiments, the multivalent chimeric molecule comprises a first siRNA and a second siRNA connected by a phosphodiester linker region. In some embodiments, the first and second siRNAs are in the same direction relative to each other, e.g., running 5′ to 3′, e.g., a serial chimeric molecule. In some embodiments, the first and second siRNAs are in the opposite direction relative to each other, e.g., one running 5′ to 3′ and the other running 3′ to 5′, e.g., an inverse chimeric molecule. In some embodiments, the multivalent chimeric molecule comprises a first strand comprising one strand of each of the first siRNA and the second siRNA connected by a linker, a first complementary strand that is at least partially complementary to the strand of the first siRNA, and a second complementary strand that is at least partially complementary to strand of the second siRNA.

In certain embodiments, the chimeric molecule comprises an IRF4 siRNA molecule targeted to human IRF4 mRNA; a c-Myc siRNA molecule targeted to human c-Myc mRNA; and; a phosphodiester linker region connecting the IRF4 siRNA and the c-Myc siRNA. The siRNAs may be formatted in serial or inverse format.

In some embodiments, the siRNA comprises, consists essentially of, or consists of a sequence at least 90% identical to one of SEQ ID NOS: 51-53, e.g., at least 90%, 91%, 92%, 93%, 94%, 95%, 96%, 97%, 98%, or 99% identical.

In some embodiments, the siRNA multivalent chimeric molecule has one or more chemical modifications. In some embodiments, the siRNA multivalent chimeric molecule is fully chemically-modified. In some embodiments, each nucleotide in the siRNA multivalent chimeric molecule is modified with a 2′-O-methyl group or a 2′-fluoro group. In some embodiments, the siRNA multivalent chimeric molecule siRNA molecules comprise at least one phosphorothioate linkage.

In some embodiments, the siRNA multivalent chimeric molecule comprises one strand comprising the sequence of SEQ ID NO:45, a first complementary strand comprising SEQ ID NO:46, and a second complementary strand comprising SEQ ID NO:47.

The invention further relates to a nucleic acid construct comprising the RNA molecule of the invention. The invention further relates to a nucleic acid construct encoding the RNA molecule of the invention and a nucleic acid construct comprising the nucleic acid molecule encoding the RNA molecule. In each of these embodiments, the nucleic acid construct may be a vector or a plasmid, e.g., an expression vector.

Another aspect of the invention relates to a composition comprising the RNA molecule, chemically-modified siRNA molecule, or nucleic acid construct of the invention and another component, e.g., a suitable carrier. In some embodiments, the composition comprises two or more of the RNA molecules, chemically-modified siRNA molecules, or nucleic acid constructs of the invention, wherein the two or more RNA molecules or chemically-modified siRNA molecule each comprise a different antisense strand. In certain embodiments, the two or more RNA molecules are present on the same nucleic acid construct, on different nucleic acid constructs or any combination thereof. In some embodiments, the composition is a pharmaceutical composition comprising the RNA molecule(s), chemically-modified siRNA molecule(s), or nucleic acid construct(s) of the invention and a pharmaceutically acceptable carrier.

It is understood that the compositions of this invention can comprise, consist essentially of, or consist of any of the RNA molecules, chemically-modified siRNA molecules, and nucleic acid constructs in any combination and in any ratio relative to one another. Furthermore, by “two or more” is meant 2, 3, 4, 5, 6, 7, 8, 9, 10, etc., up to a total number of RNA molecules, chemically-modified siRNA molecules, and nucleic acid constructs of this invention.

In some aspects of the invention, the composition or pharmaceutical composition further comprises additional components that enhance the delivery of the RNA molecule(s), chemically-modified siRNA molecule(s), or nucleic acid construct(s) of the invention to a subject, e.g., by enhancing the stability of the RNA molecule(s), chemically-modified siRNA molecule(s), or nucleic acid construct(s). In some embodiments, the additional component may be a particle, e.g., a microparticle or nanoparticle. In some embodiments, the particle is a lipid particle, e.g., a liposome, e.g., a microliposome or a nanoliposome. The liposome, microliposome, or nanoliposome may contain any components known in the art to be suitable for preparing liposomes. In some embodiments, the liposome comprises 1,2-dioleoyl-sn-glycero-3-phosphatidylcholine ( DOPC). Liposomes may be prepared by methods known in the art, e.g., as described in Pecot et al., Mol. Cancer Ther. 13:2876 (2014), incorporated by reference herein in its entirety. In some embodiments, the RNA molecule is formed into a stable nucleic acid lipid particle (SNALP), e.g., using particles such as those provided by Arbutus Biopharma (Doylestown, PA). In certain embodiments, the lipid particle comprises, consists essentially of, or consists of cholesterol, 1,2-distearoyl-sn-glycero-3-phosphocholine (DSPC), PEG-CDMA or PEG-cDSA, and 1,2-dilinoleyloxy-3-(N,N-dimethyl) aminopropane (DLinDMA) (see Judge et al., J. Clin. Invest. 119:661 (2009)). In some embodiments, the lipid particle comprises two or more of the RNA molecules or chemically-modified siRNA molecules of the invention. In some embodiments, the additional component is a targeted delivery moiety to which the RNA molecule(s), chemically-modified siRNA molecule(s), or nucleic acid construct(s) or covalently or noncovalently conjugated, e.g., ligands, aptamers, or monoclonal antibodies.

The present invention encompasses cells comprising the RNA molecules and/or nucleic acid constructs of the invention. Thus, in some embodiments, the present invention provides a transformed or transfected cell comprising a RNA molecule and/or a nucleic acid construct and/or a composition of this invention, wherein the transformed or transfected cell has reduced expression of IRF4 and/or c-Myc as compared to a control cell.

### Methods

Various methods are provided herein, employing the nucleic acid molecules, nucleic acid constructs, and/or compositions of this invention. Thus, in one aspect, the present invention provides a method of inhibiting expression of a human IRF4 gene, the method comprising contacting the cell with the RNA molecule, chemically-modified siRNA molecule, nucleic acid construct, composition, and/or pharmaceutical composition of the invention, thereby inhibiting expression of the human IRF4 gene in the cell.

Also provided herein is a method of treating cancer in a subject in need thereof, wherein the cancer expresses or over-expresses a human IRF4 gene, the method comprising delivering to the subject the RNA molecule, chemically-modified siRNA molecule, nucleic acid construct, composition, and/or pharmaceutical composition of the invention, thereby treating cancer in the subject. A cancer expressing or over-expressing a human IRF4 gene is a cancer, e.g., a tumor, in which one or more cells express or over-express the IRF4 gene. In some embodiments, the cancer also expresses or over-expresses a human c-Myc gene.

In one embodiment of each of these aspects, the subject may be one that has been diagnosed with cancer. In another embodiment, the subject may be one that is at risk of developing cancer (e.g., predisposed due to hereditary factors, smoking, viral infection, exposure to chemicals, etc.). In a further embodiment, the subject may be one that has been identified as expressing or over-expressing a IRF4 gene and has or has not been diagnosed with cancer. The cancer may be multiple myeloma or any other IRF4 associated disease, i.e., a disease driven by IRF4 expression or mutations.

The double stranded RNA or chemically-modified siRNA molecule of the invention can be delivered directly into a cell by any method known in the art, e.g., by transfection or microinjection, e.g., as part of a composition comprising lipid particles. In other embodiments, the double stranded RNA can be delivered to a subject in the form of polynucleotides encoding the RNA to produce expression of the double stranded RNA within the cells of the subject. Those skilled in the art will appreciate that the isolated polynucleotides encoding the RNAs of the invention will typically be associated with appropriate expression control sequences, e.g., transcription/translation control signals and polyadenylation signals.

It will further be appreciated that a variety of promoter/enhancer elements can be used depending on the level and tissue-specific expression desired. The promoter can be constitutive or inducible, depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest.

To illustrate, the RNA coding sequence can be operatively associated with a cytomegalovirus (CMV) major immediate-early promoter, an albumin promoter, an Elongation Factor 1-α (EF1-α) promoter, a PγK promoter, a MFG promoter, or a Rous sarcoma virus promoter.

Inducible promoter/enhancer elements include hormone-inducible and metal-inducible elements, and other promoters regulated by exogenously supplied compounds, including without limitation, the zinc-inducible metallothionein (MT) promoter; the dexamethasone (Dex)-inducible mouse mammary tumor virus (MMTV) promoter; the T7polymerase promoter system (see WO 98/10088); the ecdysone insect promoter (No et al., Proc. Natl. Acad. Sci. USA 93:3346 (1996)); the tetracycline-repressible system (Gossen et al., Proc. Natl. Acad. Sci. USA 89:5547 (1992)); the tetracycline-inducible system (Gossen et al., Science 268:1766 (1995); see also Harvey et al., Curr. Opin. Chem. Biol. 2:512 (1998)); the RU486-inducible system (Wang et al., Nat. Biotech. 15:239 (1997); Wang et al., Gene Ther., 4:432 (1997)); and the rapamycin-inducible system (Magari et al., J. Clin. Invest. 100:2865 (1997)).

Other tissue-specific promoters or regulatory promoters include, but are not limited to, promoters that typically confer tissue-specificity in neurons. These include, but are not limited to, promoters for synapsin 1, tubulin α1, platelet-derived growth factor β-chain, tyrosine hydroxylase, neuron-specific enolase, and neurofilaments. Skeletal muscle cell promoters include, but are not limited to, promoters for-actin, Pitx3, creatine kinase, and myosin light chain. Cardiac muscle cell promoters include, but are not limited to, promoters for cardiac actin, cardiac troponin T, troponin C, myosin light chain-2, and α-myosin heavy chain. Islet (beta) cell promoters include, but are not limited to, glucokinase, gastrin, insulin, and islet amyloid polypeptide.

Moreover, specific initiation signals are generally required for efficient translation of inserted RNA coding sequences. These translational control sequences, which can include the ATG initiation codon and adjacent sequences, can be of a variety of origins, both natural and synthetic.

The isolated nucleic acid encoding the double stranded RNA can be incorporated into an expression vector. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding a double stranded RNA operatively associated with the promoter, and, optionally, a termination sequence including a stop signal for RNA polymerase and a polyadenylation signal for polyadenylase.

Non-limiting examples of animal and mammalian promoters known in the art include, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, baculovirus IE1 promoter, elongation factor 1 alpha (EF1) promoter, phosphoglycerate kinase (PGK) promoter, ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), and pathogenesis and/or disease-related promoters. In addition, any of these expression sequences of this invention can be modified by addition of enhancer and/or regulatory sequences and the like.

Enhancers that may be used in embodiments of the invention include but are not limited to: an SV40 enhancer, a cytomegalovirus (CMV) enhancer, an elongation factor I (EF1) enhancer, yeast enhancers, viral gene enhancers, and the like.

Termination control regions, i.e., terminator or polyadenylation sequences, may be derived from various genes native to the preferred hosts. In some embodiments of the invention, the termination control region may comprise or be derived from a synthetic sequence, a synthetic polyadenylation signal, an SV40 late polyadenylation signal, an SV40 polyadenylation signal, a bovine growth hormone (BGH) polyadenylation signal, viral terminator sequences, or the like.

It will be apparent to those skilled in the art that any suitable vector can be used to deliver the polynucleotide to a cell or subject. The vector can be delivered to cells in vivo. In other embodiments, the vector can be delivered to cells ex vivo, and then cells containing the vector are delivered to the subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

Suitable vectors include, but are not limited to, plasmid vectors, viral vectors (e.g., retrovirus, alphavirus; vaccinia virus; adenovirus, adeno-associated virus and other parvoviruses, lentivirus, poxvirus, or herpes simplex virus), lipid vectors, poly-lysine vectors, synthetic polyamino polymer vectors, and the like.

Any viral vector that is known in the art can be used in the present invention. Protocols for producing recombinant viral vectors and for using viral vectors for nucleic acid delivery can be found in Ausubel et al., Current Protocols in Molecular Biology (Green Publishing Associates, Inc. and John Wiley & Sons, Inc., New York) and other standard laboratory manuals (e.g., Vectors for Gene Therapy. In: Current Protocols in Human Genetics. John Wiley and Sons, Inc.: 1997).

Non-viral transfer methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. For example, naked plasmids can be introduced into muscle cells by injection into the tissue. Expression can extend over many months, although the number of positive cells is typically low (Wolff et al., Science 247:247 (1989)). Cationic lipids have been demonstrated to aid in introduction of nucleic acids into some cells in culture (Felgner and Ringold, Nature 337:387 (1989)). Injection of cationic lipid plasmid DNA complexes into the circulation of mice has been shown to result in expression of the DNA in lung (Brigham et al., Am. J. Med. Sci. 298:278 (1989)). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., No Shinkei Geka 20:547 (1992); PCT publication WO 91/06309; Japanese patent application 1047381; and European patent publication EP-A-43075).

Liposomes that consist of amphiphilic cationic molecules are useful as non-viral vectors for nucleic acid delivery in vitro and in vivo (reviewed in Crystal, Science 270:404 (1995); Blaese et al., Cancer Gene Ther. 2:291 (1995); Behr et al., Bioconjugate Chem. 5:382 (1994); Remy et al., Bioconjugate Chem. 5:647 (1994); and Gao et al., Gene Therapy 2:710 (1995)). The positively charged liposomes are believed to complex with negatively charged nucleic acids via electrostatic interactions to form lipid: nucleic acid complexes. The lipid: nucleic acid complexes have several advantages as nucleic acid transfer vectors. Unlike viral vectors, the lipid: nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Felgner et al., Proc. Natl. Acad. Sci. USA 84:7413 (1987); Loeffler et al., Meth. Enzymol. 217:599 (1993); Felgner et al., J. Biol. Chem. 269:2550 (1994)).

Several groups have reported the use of amphiphilic cationic lipid: nucleic acid complexes for in vivo transfection both in animals and in humans (reviewed in Gao et al., Gene Therapy 2:710 (1995); Zhu et al., Science 261:209 (1993); and Thierry et al., Proc. Natl. Acad. Sci. USA 92:9742 (1995)). U.S. Pat. No. 6,410,049 describes a method of preparing cationic lipid: nucleic acid complexes that have a prolonged shelf life.

Nuclear localization signals can also be used to enhance the targeting of the double stranded RNA or expression vector into the proximity of the nucleus and/or its entry into the nucleus. Such nuclear localization signals can be a protein or a peptide such as the SV40large Tag NLS or the nucleoplasmin NLS. These nuclear localization signals interact with a variety of nuclear transport factors such as the NLS receptor (karyopherin alpha) which then interacts with karyopherin beta.

Expression vectors can be designed for expression of double stranded RNA in prokaryotic or eukaryotic cells. For example, double stranded RNA can be expressed in bacterial cells such as E. coli, insect cells (e.g., the baculovirus expression system), yeast cells, plant cells or mammalian cells. Some suitable host cells are discussed further in Goeddel, Gene Expression Technology: Methods in Enzymology 185, Academic Press, San Diego, Calif. (1990). Examples of bacterial vectors include, but are not limited to, pQE70,pQE60, pQE-9 (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540, and pRIT5 (Pharmacia). Examples of vectors for expression in the yeast S. cerevisiae include pYepSecl (Baldari et al., EMBO J. 6:229 (1987)), pMFa (Kurjan and Herskowitz, Cell 30:933 (1982)), pJRY88 (Schultz et al., Gene 54:113 (1987)), and pYES2 (Invitrogen Corporation, San Diego, Calif.). Non-limiting examples of baculovirus vectors available for expression of nucleic acids to produce proteins in cultured insect cells (e.g., Sf 9 cells) include the pAc series (Smith et al., Mol. Cell. Biol. 3:2156 (1983)) and the pVL series (Lucklow and Summers Virology 170:31 (1989)).

Examples of mammalian expression vectors include pWLNEO, pSV2CAT, pOG44,pXT1, pSG (Stratagene) pSVK3, PBPV, pMSG, PSVL (Pharmacia), pCDM8 (Seed, Nature 329:840 (1987)) and pMT2PC (Kaufman et al., EMBO J. 6:187 (1987)). When used in mammalian cells, the expression vector's control functions are often provided by viral regulatory elements. For example, commonly used promoters are derived from polyoma, adenovirus 2, cytomegalovirus and Simian Virus 40.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, adenovirus, geminivirus, and caulimovirus vectors. Non-limiting examples of non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

In addition to the regulatory control sequences discussed above, the recombinant expression vector can contain additional nucleotide sequences. For example, the recombinant expression vector can encode a selectable marker gene to identify host cells that have incorporated the vector.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA and RNA) into a host cell, including, but are not limited to, calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al., Molecular Cloning: A Laboratory Manual 2nd Ed. (Cold Spring Harbor, NY, 1989), and other laboratory manuals.

If stable integration is desired, often only a small fraction of cells (in particular, mammalian cells) integrate the foreign DNA into their genome. In order to identify and select integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

In one embodiment, the double stranded RNA or chemically-modified siRNA molecule of the invention is administered directly to the subject. Generally, the compounds of the invention will be suspended in a pharmaceutically-acceptable carrier (e.g., physiological saline) and administered orally, topically, or by intravenous infusion, or injected subcutaneously, intramuscularly, intracranially, intrathecally, intraperitoneally, intrarectally, intravaginally, intranasally, intragastrically, intratracheally, or intrapulmonarily. They are preferably delivered directly to the site of the disease or disorder, such as the lung, intestine, or pancreas. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the differing efficiencies of various routes of administration. For example, oral administration would be expected to require higher dosages than administration by i.v. injection (e.g., 2-, 3-, 4-, 6-, 8-, 10-; 20-, 50-, 100-, 150-, or more fold). Variations in these dosage levels can be adjusted using standard empirical routines for optimization as is well understood in the art. Administrations can be single or multiple. Encapsulation of the inhibitor in a suitable delivery vehicle (e.g., polymeric microparticles or implantable devices) may increase the efficiency of delivery, particularly for oral delivery.

According to certain embodiments, the double stranded RNA or chemically-modified siRNA molecule can be targeted to specific cells or tissues in vivo. Targeting delivery vehicles, including liposomes and viral vector systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., Biochemistry 25:5500 (1986); Ho et al., J. Biol. Chem. 262:13979 (1987); Ho et al., J. Biol. Chem. 262:13973 (1987); and U.S. Pat. No. 4,957,735 to Huang et al., each of which is incorporated herein by reference in its entirety). Enveloped viral vectors can be modified to deliver a nucleic acid molecule to a target cell by modifying or substituting an envelope protein such that the virus infects a specific cell type. In adenoviral vectors, the gene encoding the attachment fibers can be modified to encode a protein domain that binds to a cell-specific receptor. Herpesvirus vectors naturally target the cells of the central and peripheral nervous system. Alternatively, the route of administration can be used to target a specific cell or tissue. For example, intracoronary administration of an adenoviral vector has been shown to be effective for the delivery of a gene to cardiac myocytes (Maurice et al., J. Clin. Invest. 104:21 (1999)). Intravenous delivery of cholesterol-containing cationic liposomes has been shown to preferentially target pulmonary tissues (Liu et al., Nature Biotechnol. 15:167 (1997)), and effectively mediate transfer and expression of genes in vivo. Other examples of successful targeted in vivo delivery of nucleic acid molecules are known in the art. Finally, a recombinant nucleic acid molecule can be selectively (i.e., preferentially, substantially exclusively) expressed in a target cell by selecting a transcription control sequence, and preferably, a promoter, which is selectively induced in the target cell and remains substantially inactive in non-target cells.

The double stranded RNA or chemically-modified siRNA molecule of the present invention can optionally be delivered in conjunction with other therapeutic agents. The additional therapeutic agents can be delivered concurrently with the double stranded RNA or chemically-modified siRNA molecule of the invention. As used herein, the word “concurrently” means sufficiently close in time to produce a combined effect (that is, concurrently can be simultaneously, or it can be two or more events occurring within a short time period before or after each other). In one embodiment, the double stranded RNA or chemically-modified siRNA molecule of the invention are administered in conjunction with agents useful for treating cancer, such as: 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide). In another embodiment, the compounds of the invention are administered in conjunction with anti-angiogenesis agents, such as antibodies to VEGF (e.g., bevacizumab (AVASTIN), ranibizumab (LUCENTIS)) and other promoters of angiogenesis (e.g., bFGF, angiopoietin-1), antibodies to alpha-v/beta-3 vascular integrin (e.g., VITAXIN), angiostatin, endostatin, dalteparin, ABT-510, CNGRC peptide TNF alpha conjugate, cyclophosphamide, combretastatin A4 phosphate, dimethylxanthenone acetic acid, docetaxel, lenalidomide, enzastaurin, paclitaxel, paclitaxel albumin-stabilized nanoparticle formulation (Abraxane), soy isoflavone (Genistein), tamoxifen citrate, thalidomide, ADH-1 (EXHERIN), AG-013736,AMG-706, AZD2171, sorafenib tosylate, BMS-582664, CHIR-265, pazopanib, PI-88, vatalanib, everolimus, suramin, sunitinib malate, XL184, ZD6474, ATN-161, cilenigtide, and celecoxib, or any combination thereof.

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers.

### Pharmaceutical Compositions

As a further aspect, the invention provides pharmaceutical formulations and methods of administering the same to achieve any of the therapeutic effects (e.g., treatment of cancer) discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The double stranded RNA, chemically-modified siRNA molecule, or nucleic acid construct of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the double stranded RNA or chemically-modified siRNA molecule (including the physiologically acceptable salts thereof) is typically admixed with, inter alia, an acceptable carrier. The carrier can be a solid or a liquid, or both, and is preferably formulated with the double stranded RNA or chemically-modified siRNA molecule as a unit-dose formulation, for example, a tablet, which can contain from 0.01 or 0.5% to 95% or 99% by weight of the double stranded RNA or chemically-modified siRNA molecule. One or more double stranded RNAs or chemically-modified siRNA molecules can be incorporated in the formulations of the invention, which can be prepared by any of the well-known techniques of pharmacy.

A further aspect of the invention is a method of treating subjects in vivo, comprising administering to a subject a pharmaceutical composition comprising a double stranded RNA or chemically-modified siRNA molecule of the invention in a pharmaceutically acceptable carrier, wherein the pharmaceutical composition is administered in a therapeutically effective amount. Administration of the double stranded RNA or chemically-modified siRNA molecule of the present invention to a human subject or an animal in need thereof can be by any means known in the art for administering compounds.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site). In some embodiments, the formulation can be delivered locally to ischemic tissue. In certain embodiments, the formulation can be a slow release formulation, e.g., in the form of a slow release depot.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In particular embodiments, the compound is administered to the subject in a therapeutically effective amount, as that term is defined above. Dosages of pharmaceutically active compounds can be determined by methods known in the art, see, e.g., Remington's Pharmaceutical Sciences (Maack Publishing Co., Easton, Pa). The therapeutically effective dosage of any specific compound will vary somewhat from compound to compound, and patient to patient, and will depend upon the condition of the patient and the route of delivery. As a general proposition, a dosage from about 0.001 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the compound for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of cancer. In certain embodiments, the subject has or is at risk for cancer.

The following examples are not intended to limit the scope of the claims to the invention, but are rather intended to be exemplary of certain embodiments. Any variations in the exemplified methods that occur to the skilled artisan are intended to fall within the scope of the present invention. As will be understood by one skilled in the art, there are several embodiments and elements for each aspect of the claimed invention, and all combinations of different elements are hereby anticipated, so the specific combinations exemplified herein are not to be construed as limitations in the scope of the invention as claimed. If specific elements are removed or added to the group of elements available in a combination, then the group of elements is to be construed as having incorporated such a change.

### EXAMPLE 1

### Design of IRF4 siRNA

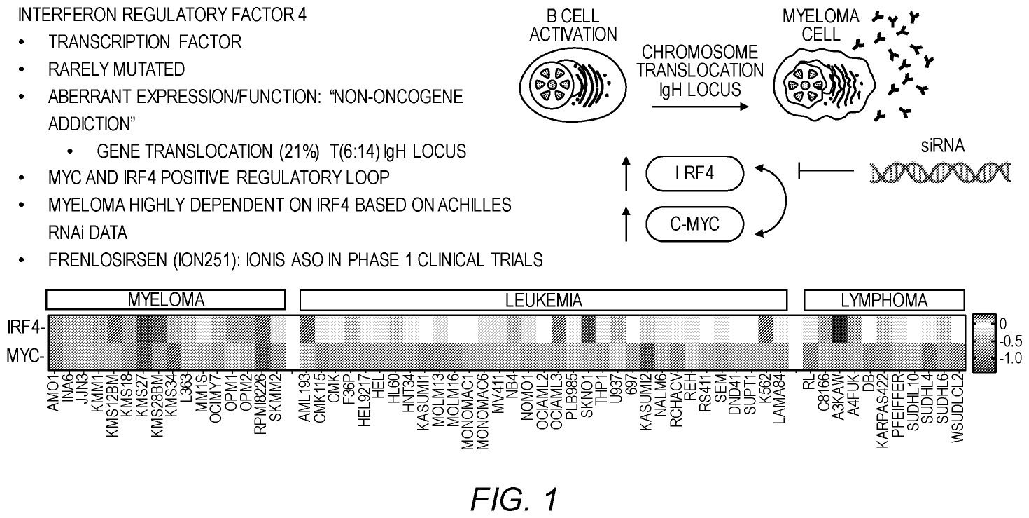

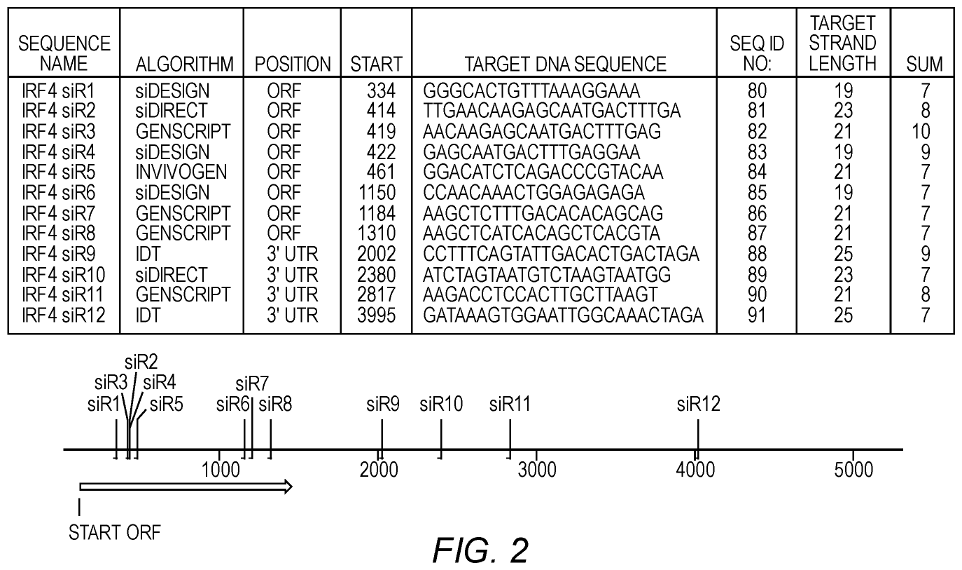

IRF4 is often overexpressed in plasma cells that have become malignant (i.e., evolved into MM), and preclinical models have demonstrated that suppressing IRF4 reduces the viability of MM cells in vitro (FIG. 1). The heatmap at the bottom of FIG. 1 shows Demeter2 dependency data revealing that MM cells are often highly dependent on IRF4 (the more negative the value, the more dependent). To develop therpaeutic agants to inhibit IRF4 expression, the full IRF4 sequence (ORF+UTRs) was put into multiple online databases to design siRNAs. However, there was very little overlap between the results from different databases. Thus, a scoring system/rank variables was created including variables such as GC content (30-55%), low BLAST hits, appearing in multiple databases, and homology with mouse IRF4 sequence. 12 siRNAs were selected with the largest SUMs>6. The selected siRNAs target sequences both in the ORF and UTR (FIG. 2). The sequences are shown in Table 3.

TABLE 3
siRNA sequences

| | S | Sense | | |
|---|---|---|---|---|
| | | Strand | | |
| | | Sequence | | |
| | | (5′ to 3′) | | |
| | A | Antisense | | |
| | | Strand | | SEQ |
| Oligo | | Sequence | | ID |
| Name | | (5′ to 3′) | Overhang | NO |
| IRF4 | S | GGGCACUGUUU | [dT][dT] | 1 |
| siR1 | | AAAGGAAA | | |
| | A | UUUCCUUUAAA | [dT][dT] | 2 |
| | | CAGUGCCC | | |
| IRF4 | S | GAACAAGAGCA | GA | 3 |
| siR2 | | AUGACUUU | | |
| | A | AAAGUCAUUGC | AA | 4 |
| | | UCUUGUUC | | |
| IRF4 | S | AACAAGAGCAA | [dT][dT] | 5 |
| siR3 | | UGACUUUGAG | | |
| | A | CUCAAAGUCAU | [dT][dT] | 6 |
| | | UGCUCUUGUU | | |
| IRF4 | S | GAGCAAUGACU | [dT][dT] | 7 |
| siR4 | | UUGAGGAA | | |
| | A | UUCCUCAAAGU | [dT][dT] | 8 |
| | | CAUUGCUC | | |
| IRF4 | S | GGACAUCUCAG | [dT][dT] | 9 |
| siR5 | | ACCCGUACAA | | |
| | A | UUGUACGGGUC | [dT][dT] | 10 |
| | | UGAGAUGUCC | | |
| IRF4 | S | CCAACAAACUG | [dT][dT] | 11 |
| siR6 | | GAGAGAGA | | |
| | A | UCUCUCUCCAG | [dT][dT] | 12 |
| | | UUUGUUGG | | |
| IRF4 | S | AAGCUCUUUGA | [dT][dT] | 13 |
| siR7 | | CACACAGCAG | | |
| | A | CUGCUGUGUGU | [dT][dT] | 14 |
| | | CAAAGAGCUU | | |
| IRF4 | S | AAGCUCAUCAC | [dT][dT] | 15 |
| siR8 | | AGCUCACGUA | | |
| | A | UACGUGAGCUG | [dT][dT] | 16 |
| | | UGAUGAGCUU | | |
| IRF4 | S | CCUUUCAGUAU | [dT][dT] | 17 |
| siR9 | | UGACACUGACU | | |
| | | AGA | | |
| | A | UCUAGUCAGUG | CA | 18 |
| | | UCAAUACUGAA | | |
| | | AGG | | |
| IRF4 | S | CUAGUAAUGUC | [dT][dT] | 19 |
| siR10 | | UAAGUAAU | | |
| | A | AUUACUUAGAC | AU | 20 |
| | | AUUACUAG | | |
| IRF4 | S | AAGACCUCCAC | GG | 21 |
| siR11 | | UUGCUUAAGU | | |
| | A | ACUUAAGCAAG | [dT][dT] | 22 |
| | | UGGAGGUCUU | | |
| IRF4 | S | GAUAAAGUGGA | [dT][dT] | 23 |
| siR12 | | AUUGGCAAACU | | |
| | | AGA | | |
| | A | UCUAGUUUGCC | AA | 24 |
| | | AAUUCCACUUU | | |
| | | AUC | | |

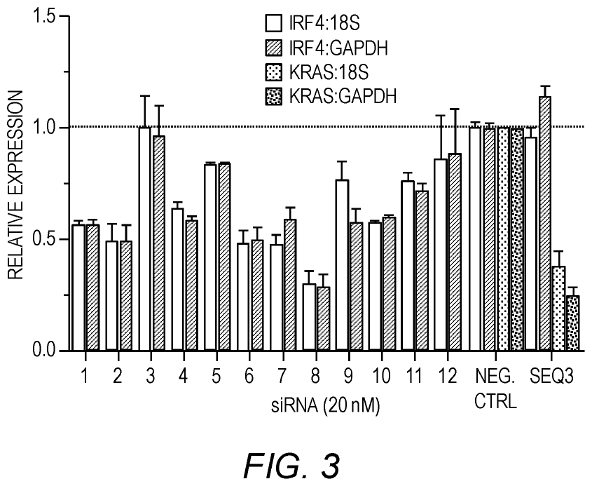

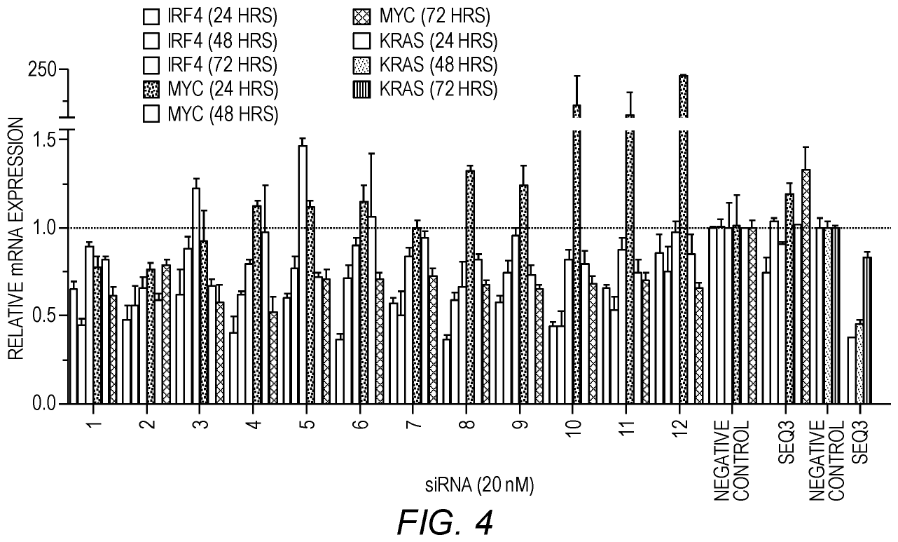

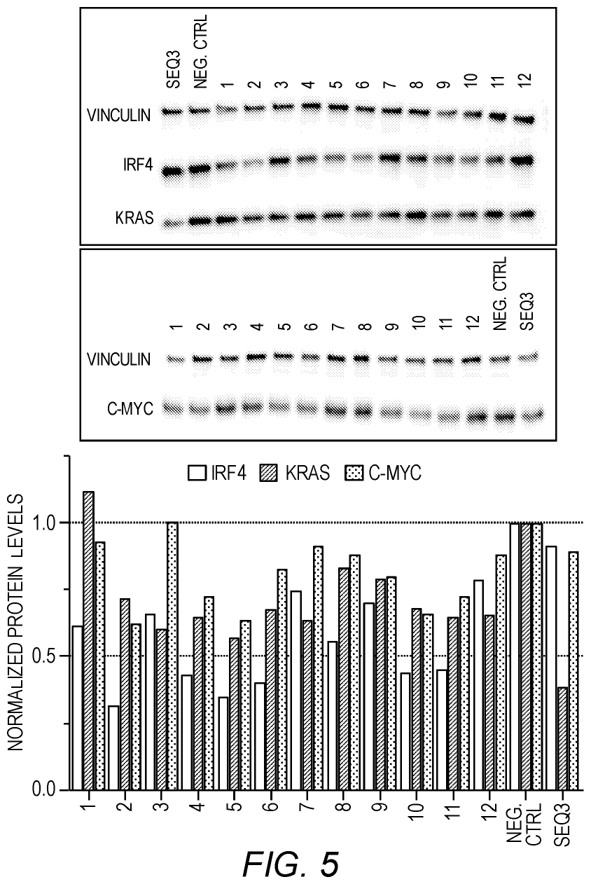

The 12 siRNAs were tested in RPMI 8226 myeloma cells for inhibition of IRF4 expression. The results showed that almost all of the siRNAs were capable of inhibiting IRF4 expression (FIG. 3). Additionally, it was evaluated whether select, fully chemically-modified siRNAs were capable of inhibiting both IRF4 and c-Myc expression over time (FIG. 4). Decreased protein levels of IRF4 and c-Myc were detected by immunoblotting (FIG. 5).

### EXAMPLE 2

### Synthetic Chemically Modified siRNAs Decrease c-IRF4 Protein Expression

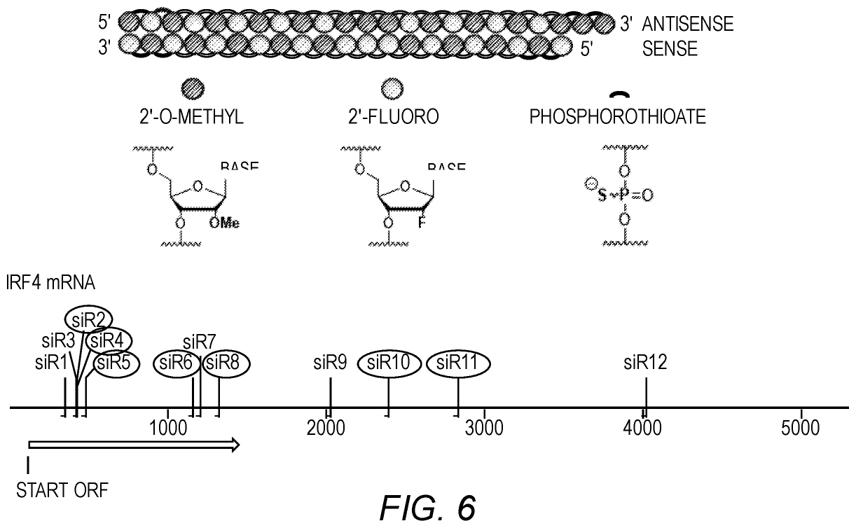

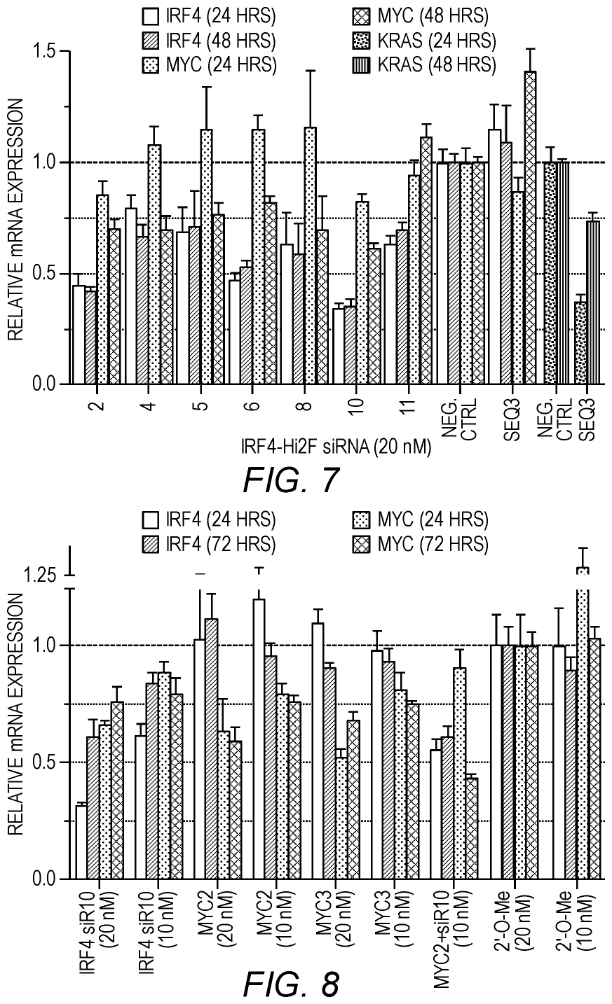

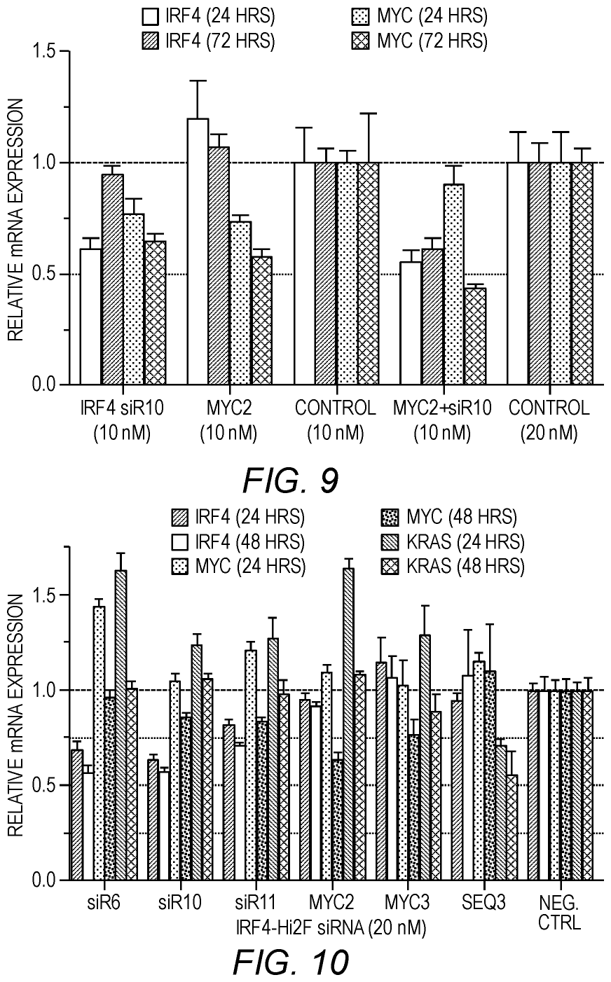

Of the 12 IRF4 siRNAs, 7 were selected to be fully chemically modified with a Hi2F pattern, which consists of roughly a 50/50 mixture of 2′-fluoro (2′F) and 2′-O-methyl (2′OMe) ribose modifications (FIG. 6). The sequences are disclosed in Table 4. The Hi2F siRNAs were capable of decreasing IRF4 and c-Myc mRNA levels ove time in RPMI-8229 cells (FIG. 7), consistent with IRF4 having a feedforward regulatory loop on c-Myc expression. Further, siRNAs to IRF4, c-Myc, or the combination resulted in decreased IRF4and c-Myc mRNA levels (FIG. 8) Key data from this experiment are shown in FIG. 9. Taken together, these data demonstrate several effective chemically-modified IRF4 siRNAs with a Hi2F pattern have been identified.

TABLE 4
Fully modified siRNA sequences

| | | | SEQ |
|---|---|---|---|
| | | Sequence | ID |
| Name | Strand | (5′-3′) | NO |
| IRF4 | S | [2flU]*[mU]*[2flG] | 25 |
| siR2 | | [mA][2flA][mC][2flA] | |
| Hi2F | | [mA][2flG][2flA] | |
| | | [2flG][mC][2flA][mA] | |
| | | [2flU][mG][2flA][mC] | |
| | | [2flU]*[mU]*[2flU] | |
| | AS | [mA]*[2flA]*[mA] | 26 |
| | | [2flG][mU][2flC][mA] | |
| | | [2flU][mU][2flG][mC] | |
| | | [mU][mC][2flU][mU] | |
| | | [2flG][mU][2flU][mC] | |
| | | [sflA][mA]*[mA]*[mG] | |
| IRF4 | S | [2flA]*[mA]*[2flG] | 27 |
| siR4 | | [mA][2flG][mC][2flA] | |
| Hi2F | | [mA][2flU][2flG] | |
| | | [2flA][mC][2flU][mU] | |
| | | [2flU][mG][2flA][mG] | |
| | | [2flG]*[mA]*[2flA] | |
| | AS | [mU]*[2flU]*[mC] | 28 |
| | | [2flC][mU][2flC] | |
| | | [mA][2flA][mA][2flG] | |
| | | [mU][mC][mA][2flU] | |
| | | [mU][2flG][mC][2flU] | |
| | | [mC][23flU][mU]*[mG]* | |
| | | [mU] | |
| IRF4 | S | [2flG]*[mG]*[2flA] | 29 |
| siR5 | | [mC][2flA][mU][2flC] | |
| Hi2F | | [mU][2flC][2flA] | |
| | | [2flG][mA][2flC] | |
| | | [mC][2flC][mG][2flU] | |
| | | [mA][2flC]*[mA]* | |
| | | [2flA] | |
| | AS | [mU]*[2flU]*[mG] | 30 |
| | | [2flU][mA][2flC][mG] | |
| | | [2flG][mG][2flU][mC] | |
| | | [mU][mG][2flA][mG] | |
| | | [2flA][mU][2flG][mU] | |
| | | [2flC][mC]*[mA]*[mG] | |
| IRF4 | S | [2flG]*[mC]*[2flC] | 31 |
| siR6 | | [mC][2flA][mA][2flC] | |
| Hi2F | | [mA][2flA][2flA] | |
| | | [2flC][mU][2flG][mG] | |
| | | [2flA][mG][2flA][mG] | |
| | | [2flA]*[mG]*[2flA] | |
| | AS | [mU]*[2flC]*[mU] | 32 |
| | | [2flC][mU][2flC][mU] | |
| | | [2flC][mC][2flA][mG] | |
| | | [mU][mU][2flU][mG] | |
| | | [2flU][mU][2flG][mG] | |
| | | [2flG][mC]*[mC]*[mG] | |
| IRF4 | S | [2flA]*[mA]*[2flG] | 33 |
| siR8 | | [mC][2flU][mC][2flA] | |
| Hi2F | | [mU][2flC][2flA] | |
| | | [2flC][mA][2flG] | |
| | | [mC][2flU][mC][2flA] | |
| | | [mC][2flG]*[mU]* | |
| | | [2flA] | |
| | AS | [mU]*[2flA]*[mC] | 34 |
| | | [2flG][mU][2flG][mA] | |
| | | [2flG][mC][2flU][mG] | |
| | | [mU][mG][2flA][mU] | |
| | | [2flG][mA][2flG][mC] | |
| | | [2flU][mU]*[mU]*[mC] | |
| IRF4 | S | [2flA]*[mU]*[2flC] | 35 |
| siR10 | | amU][2flA][mG][2flU] | |
| Hi2F | | [mA][2flA][2flU] | |
| | | [2flG][mU][2flC][mU] | |
| | | [2flA][mA][2flG][mU] | |
| | | [2flA]*[mA]*[2flU] | |
| | AS | [mA]*[2flU]*[mU] | 36 |
| | | [2flA][mC][2flU][mU] | |
| | | [2flA][mG][2flA][mC] | |
| | | [mA][mU][2flU][mA] | |
| | | [2flC][mU][2flA][mG] | |
| | | [2flA][mU]*[mU]*[mG] | |
| IRF4 | S | [2flA]*[mA]*[2flG] | 37 |
| siR11 | | [mA][2flC][mC][2flU] | |
| Hi2F | | [mC][2flC][2flA] | |
| | | [2flC][mU][2flU][mG] | |
| | | [2flC][mU][2flU][mA] | |
| | | [2flA]*[mG]*[2flU] | |
| | AS | [mA]*[2flC]*[mU] | 38 |
| | | [2flU][mA][2flA][mG] | |
| | | [2flC][mA][2flA][mG] | |
| | | [mU][mG][2flG][mA] | |
| | | [2flG][mG][2flU][mC] | |
| | | [2flU][mU]*[mU]*[mG] | |
| IRF4 | S | [mG]*[mC]*[mC][mC] | 39 |
| siR6 | | [mA][mA][2flC][mA] | |
| HiOMe | | [2flA][2flA][2flC] | |
| | | [mU][mG][mG][mA][mG] | |
| | | [mA][mG][mA]*[mG]* | |
| | | [mA] | |
| | AS | [mU]*[2flC]*[mU][mC] | 40 |
| | | [mU][2flC][mU][mC] | |
| | | [mC][mA][mG][mU] | |
| | | [mU][2flU][mG][2flU] | |
| | | [mU][mG][mG][mG][mC] | |
| | | *[mC]*[mG] | |
| IRF4 | S | [mA]*[mU]*[mC][mU] | 41 |
| siR10 | | [mA][mG][2flU][mA] | |
| HiOMe | | [2flA][2flU][2flG] | |
| | | [mU][mC][mU][mA] | |
| | | [mA][mG][mU][mA]* | |
| | | [mA]*[mU] | |
| | AS | [mA]*[2flU]*[mU] | 42 |
| | | [mA][mC][2flu][mU] | |
| | | [mA][mG][mA][mC][mA] | |
| | | [mU][2flU][mA][2flC] | |
| | | [mU][mA][mG][mA][mU] | |
| | | *[mU]*[mG] | |
| IRF4 | S | [mA]*[mA]*[mG][mA] | 43 |
| siR11 | | [mC][mC][2flU][mC] | |
| HiOMe | | [2flC][2flA][2flC] | |
| | | [mU][mU][mG][mC] | |
| | | [mU][mU][mA][mA]* | |
| | | [mG]*[mU] | |
| | AS | [mA]*[2flC]*[mU] | 44 |
| | | [mU][mA][2flA][mG] | |
| | | [mC][mA][mA][mG][mU] | |
| | | [mG][2flG][mA][2flG] | |
| | | [mG][mU][mC][mU][mU] | |
| | | *[mU]*[mG] | |

S - sense strand
AS - antisense strand
m - 2′-O-methyl on sugar moieties
2fl - 2′-fluoro on sugar moieties
* - phosphorothioate in between nucleotides

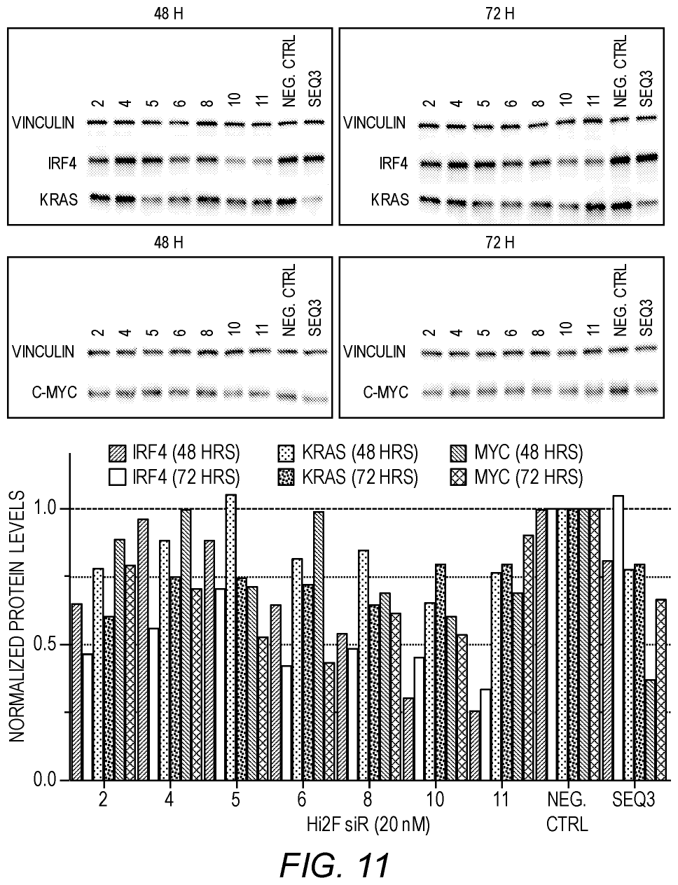

Similar experiments with KMS-11 myeloma cells demonstrated that siRNAs to IRF4 and c-Myc decreased IRF4 and c-Myc mRNA levels over time (FIG. 10). The decrease wa reflected in decreased protein levels over time (FIG. 11). These data demonstrate the IRF4 siRNAs work in several MM cell lines.

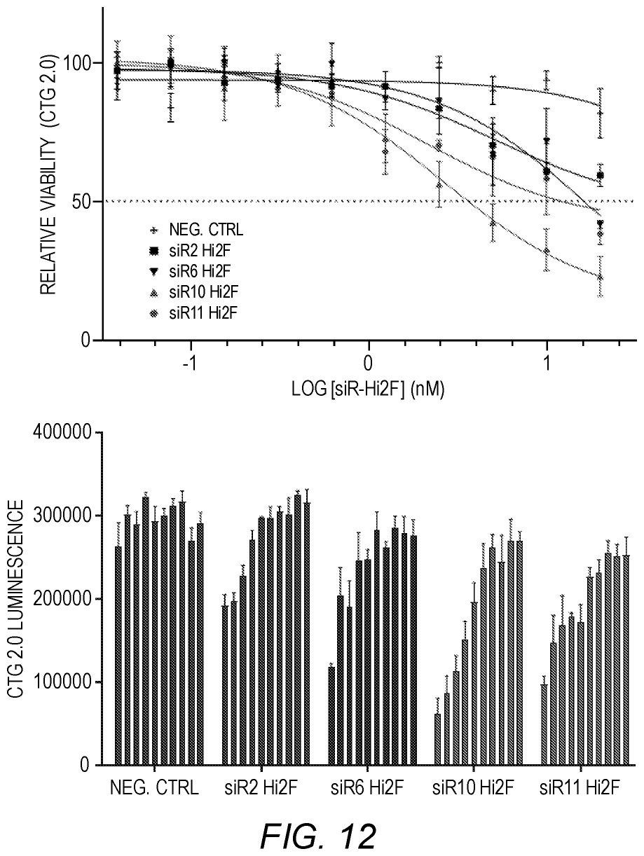

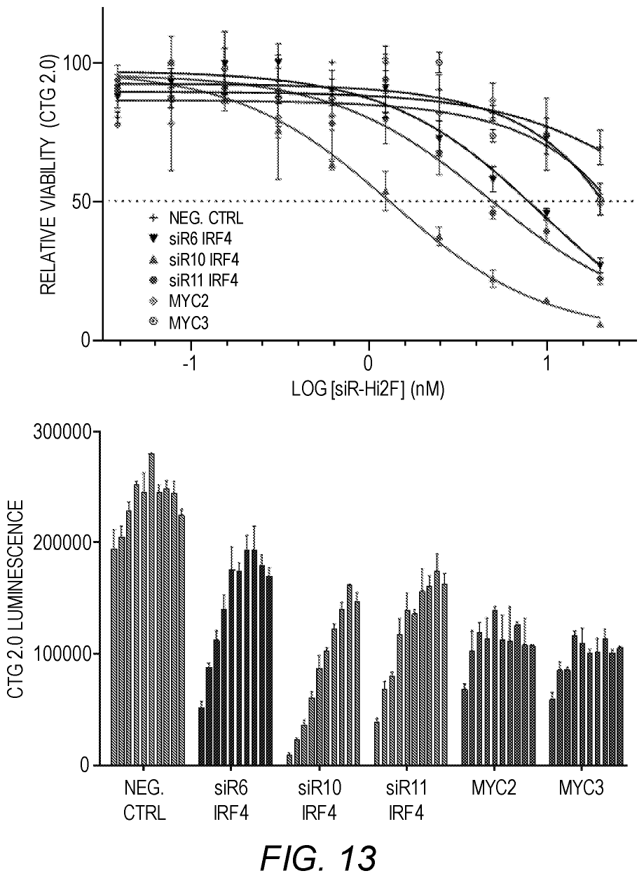

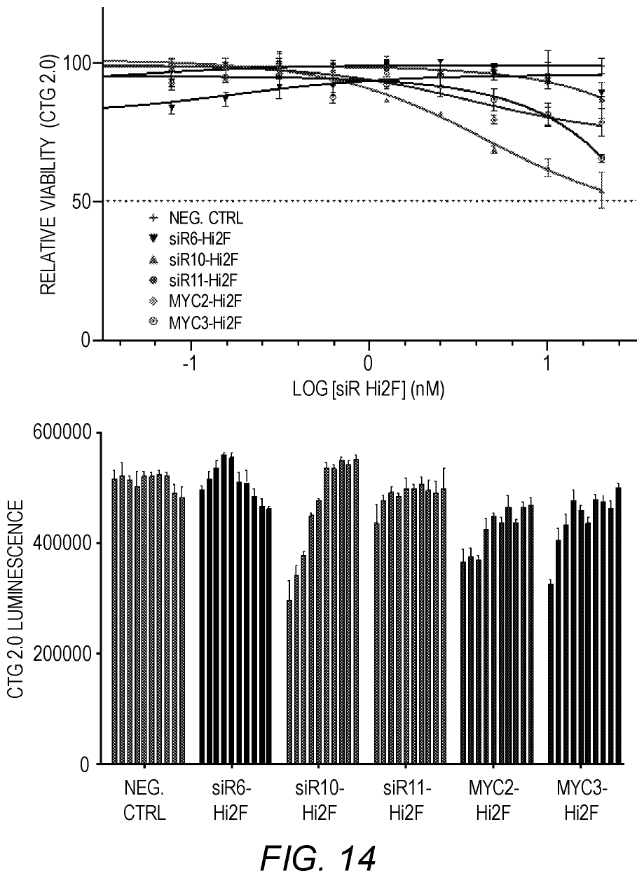

The effect of IRF4 siRNAs on cell viability was tested in RPMI-8226 cells. The results show that cell viability at 5 days decreased in a dose-dependent manner (FIG. 12). c-Myc siRNAs showed a similar effect on cell viability (FIG. 13). Similar decreases in cell viability were seen with IRF4 siRNAs in KMS-11 cells (FIG. 14).

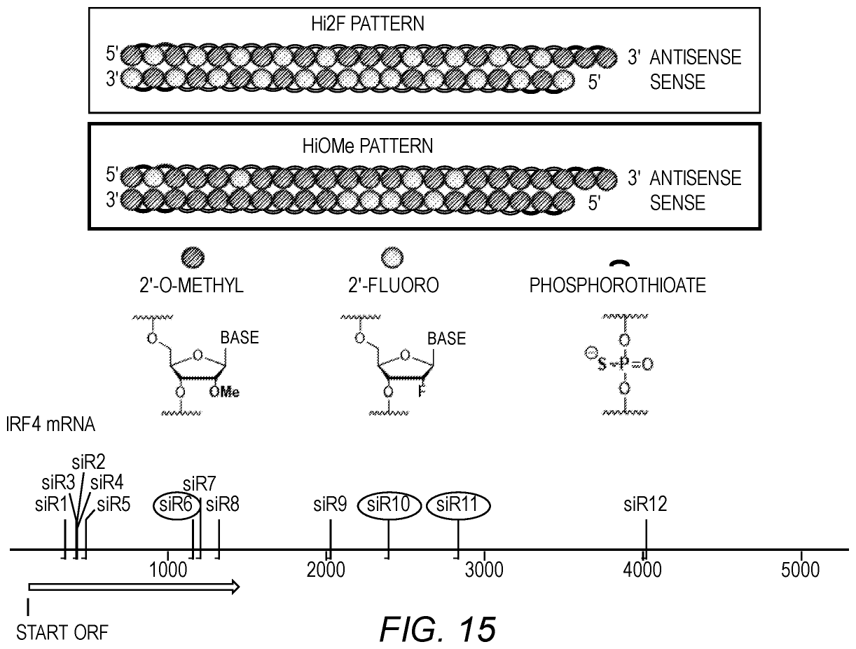

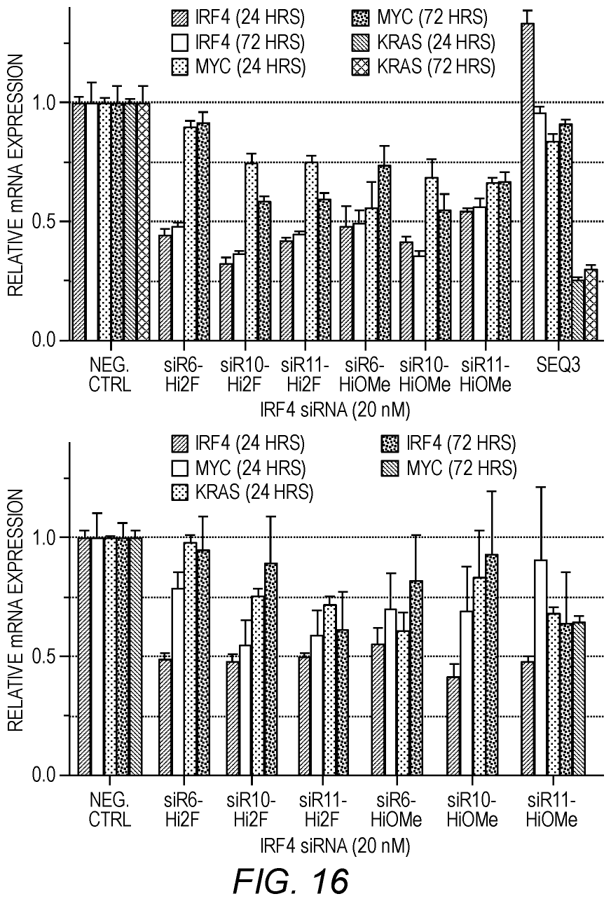

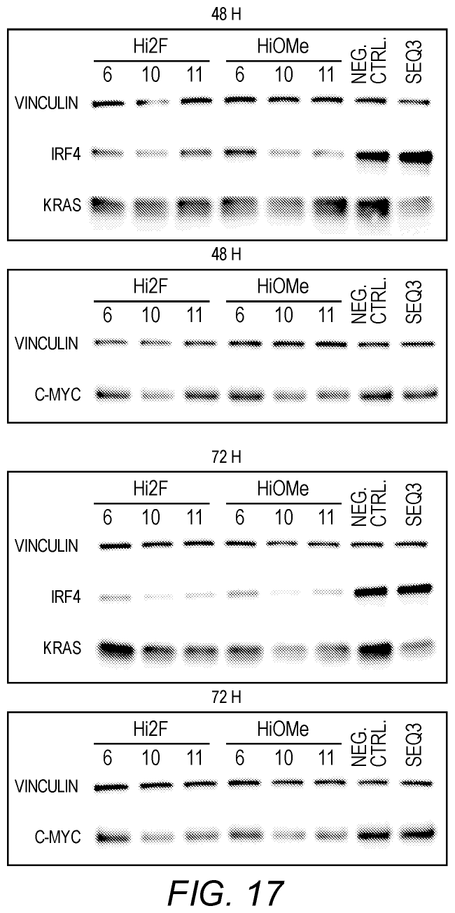

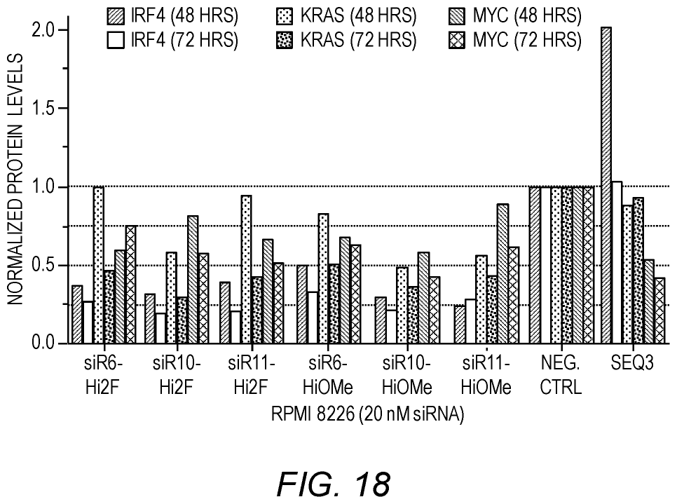

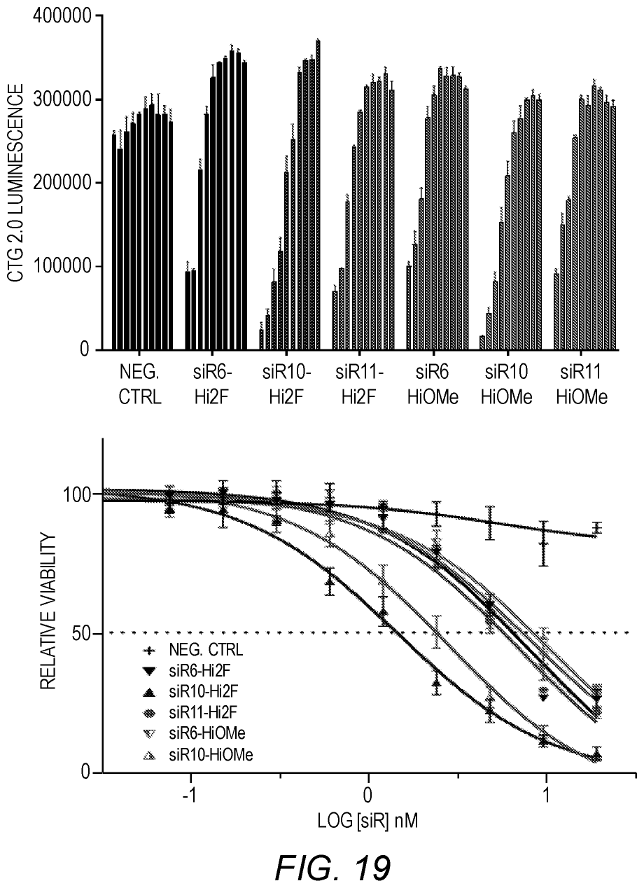

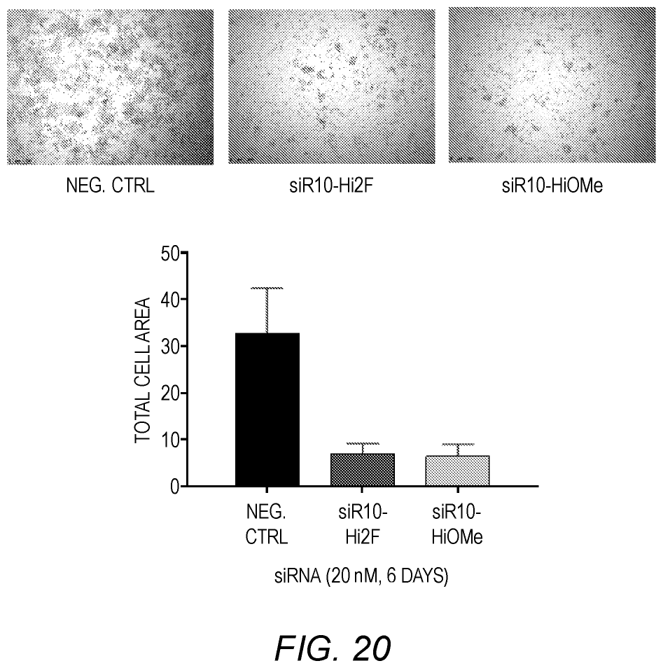

Of the 12 IRF4 siRNAs, 3 were selected to be fully chemically modified with a HiOMe pattern, which consists of a majority of 2′OMe ribose modifications and a minimum of 2′F modifications in order to improve in vivo stability and avoid nuclease degradation (FIG. 15). The sequences are disclosed in Table 4. The HiOMe modified siRNAs had a similar effect on siRNA efficacy as the Hi2F modifications (FIG. 16). The similarity was also seen with protein expression (FIGS. 17 and 18) and cell viability (FIGS. 19 and 20 and Table 5). These data demonstrate a more stabilized HiOMe pattern still has high levels of activity in silencing IRF4 on a transcript and protein level, and in inhibiting cell viability.

TABLE 5

| siR | GI50 (nM) |
|---|---|
| siR6-Hi2F | 6.7 |
| siR10-Hi2F | 1.5 |
| siR11-Hi2F | 5.9 |
| siR6-HiOMe | 7.6 |
| siR10-HiOMe | 2.5 |
| siR11-HiOMe | 8.7 |

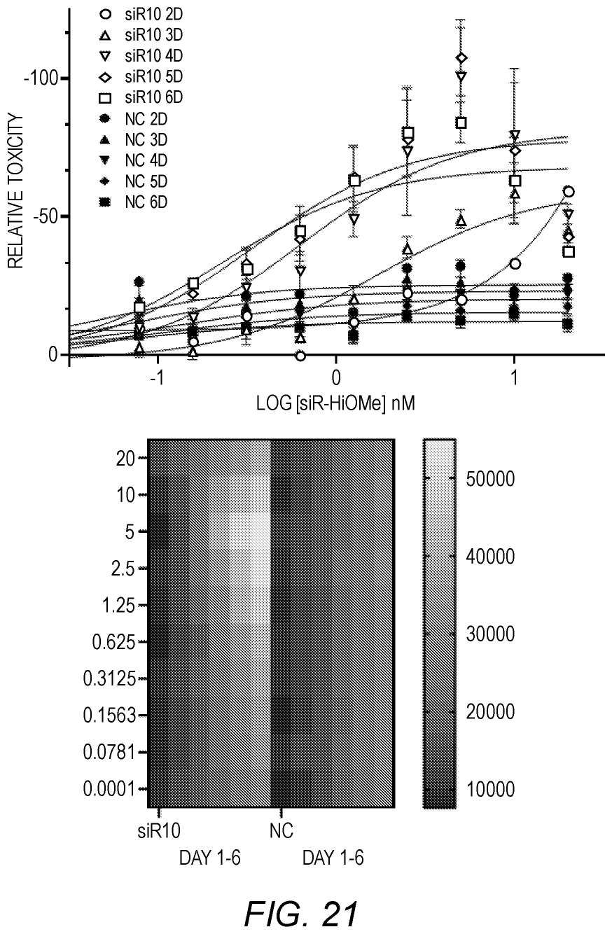

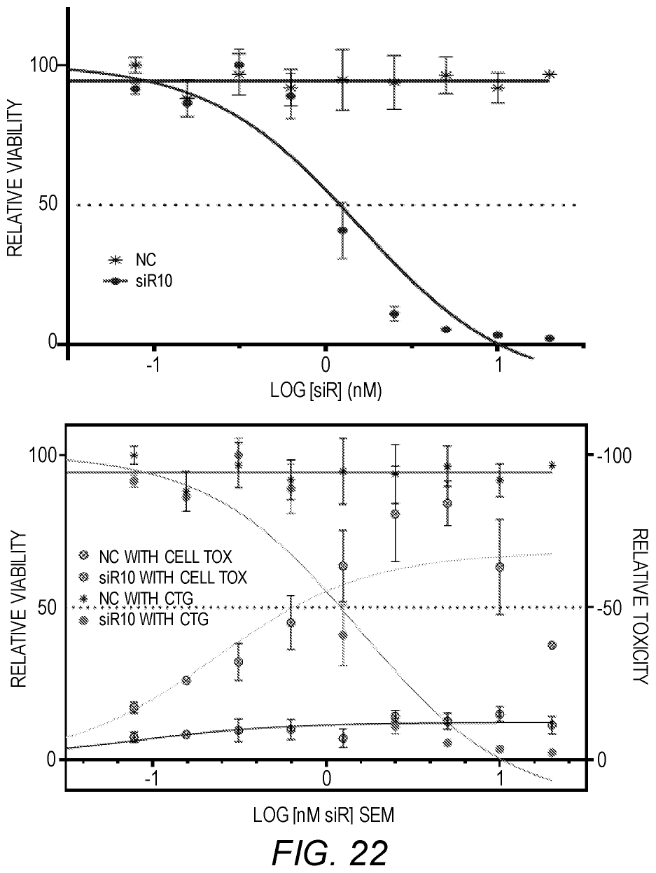

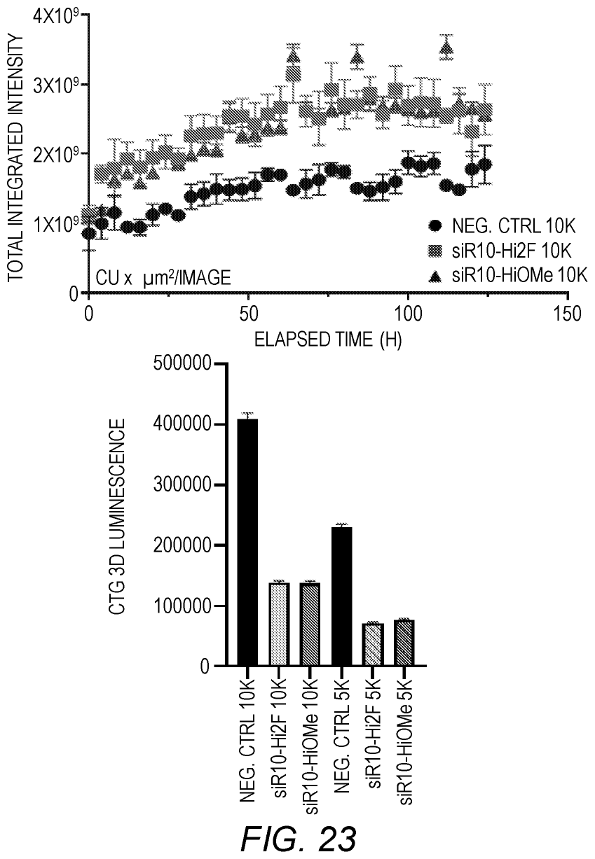

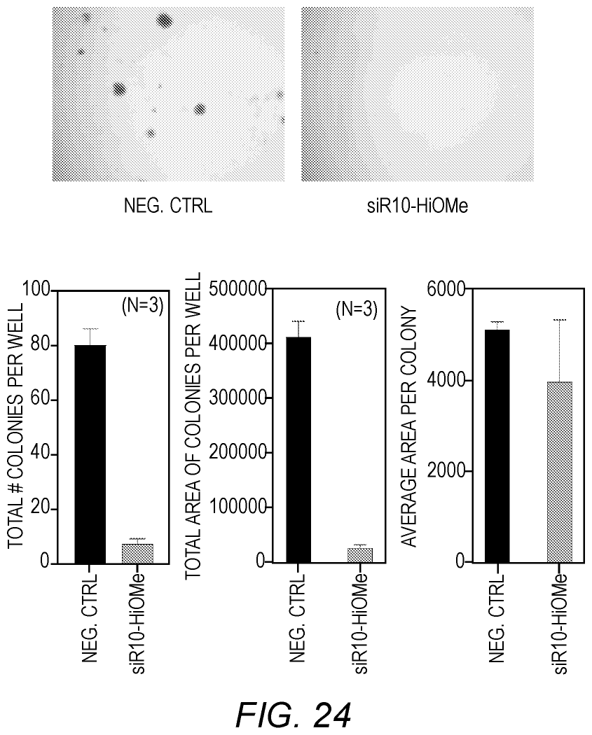

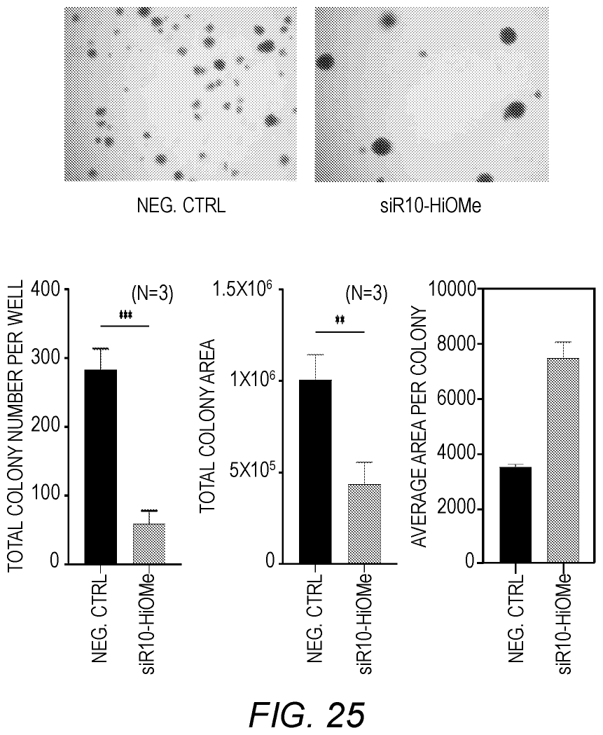

IRF4 siR10-HiOMe was demonstrated to create cellular toxicity using apoptosis and cell proliferation assays in RPMI-8226 cells (FIGS. 21 and 22). Similarly, IRF4 siR 10 Hi2F and HiOMe siRNAs increased cellular toxicity and decreased viability in spheroids of RPMI-8226 cells (FIG. 23). IRF4 siR10-HiOMe also inhbited agarose colony formation of RPMI-8226 cells (FIG. 24). These data demonstrate that the IRF4 siR 10 Hi2F and HiOMe compositions have potent activity in inhibiting MM phenotypes.

### EXAMPLE 3

### Inverted Chimera Combining c-Myc and IRF4-Targeting siRNAs

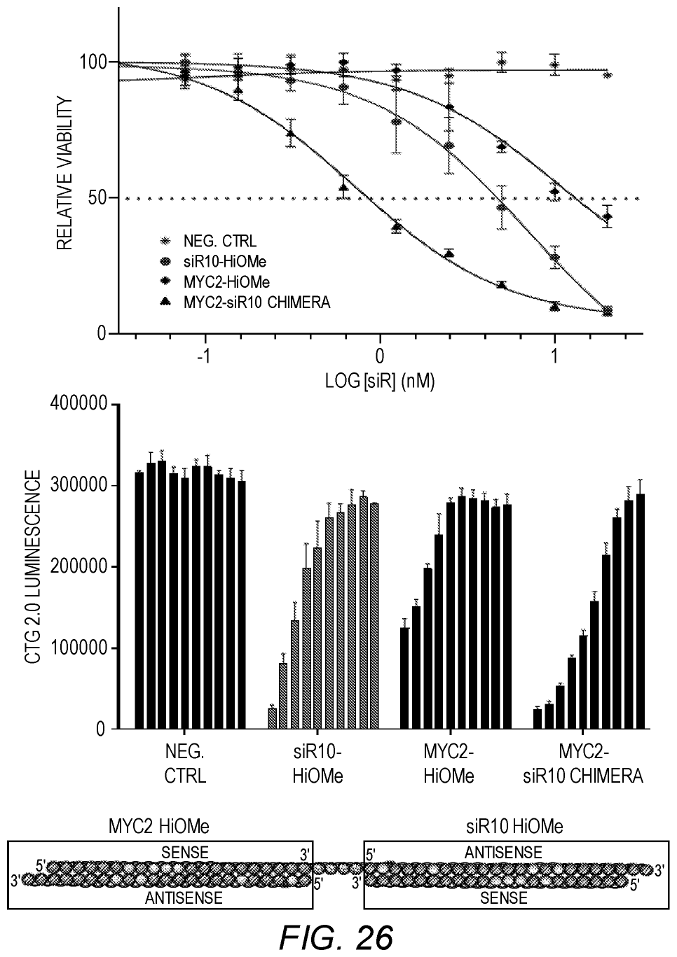

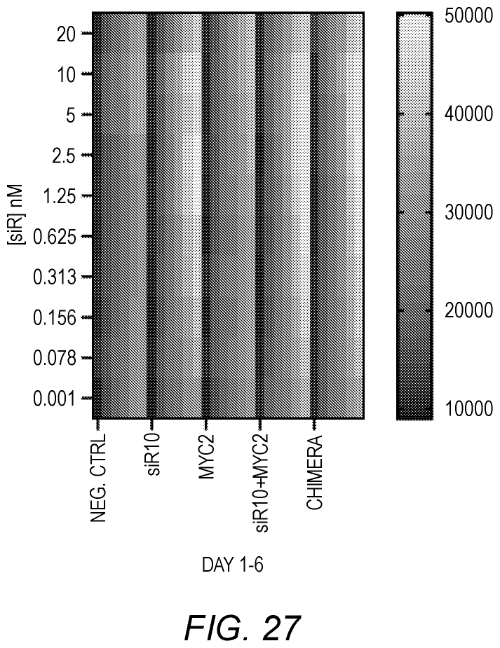

An inverted chimera comprising a Myc HiOMe siRNA linked to IRF4 siR 10 HiOMe siRNA separated by a cleavable DNA bridge was designed (FIG. 26 and Table 6). The chimera had a significantly improved effect on viability of RPMI-8226 cells compared to the individual siRNAs (FIG. 26 and Table 7), producing a significant leftward shift in GIsos indicating a substantial improvement in potency. The chimera produced cellular toxicity in RPMI-8226 cells (FIG. 27). Taken together, the inverted chimera that co-targets Myc and IRF4 has synergistic activity in targeted MM cells.

TABLE 6
Fully modified chimera siRNA sequences

| | | | SEQ |
|---|---|---|---|
| | | Sequence | ID |
| Name | Strand | (5′-3′) | NO |
| MYC2- | S | [mA]*[mC]*[mG][mA] | 45 |
| HiOMe/ | | [mG][mA][2flA][mC] | |
| IRF4 | | [2flA][2flG][2flU] | |
| siR10- | | [mU][mG][mA][mA] | |
| HiOMe | | [mA][mC][mA][mC] | |
| | | *[mA]*[mA]TTTT[mA] | |
| | | *[2flU]*[mU][mA] | |
| | | [mC][2flU][mU][mA] | |
| | | [mG][mA][mC][mA] | |
| | | [mU][2flU][mA] | |
| | | [2flC][mU][mA][mG] | |
| | | [mA][mU]*[mU]*[mG] | |
| | Myc2 | [mU]*[2flU]*[mG] | 46 |
| | AS | [mU][mG][2flU][mU] | |
| | | [mU][mC][mA][mA] | |
| | | [mC][mU][2flG][mU] | |
| | | [2flU][mC][mU][mC] | |
| | | [mG][mU]*[mC]*[mG] | |
| | IRF4 | [mA]*[mU]*[mC][mU] | 47 |
| | siR10 | [mA][mG][2flU][mA] | |
| | S | [2flA][2flU][2flG] | |
| | | [mU][mC][mU][mA] | |
| | | [mA][mG][mU][mA] | |
| | | *[mA]*[mU] | |

S - sense strand
AS - antisense strand
m - 2′-O-methyl on sugar moieties
2fl - 2′-fluoro on sugar moieties
* - phosphorothioate in between nucleotides

TABLE 7

| siR | GI₅₀ (nM) |
|---|---|
| siR10 | 4.744 |
| Myc2 | 13.21 |
| Chimera | 0.8685 |

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. A siRNA molecule targeted to a human interferon regulatory factor-4 (IRF4) mRNA, wherein the siRNA molecule comprises at least one chemical modification, and wherein the siRNA molecule comprises one of the following pairs of sequences:
sense strand of SEQ ID NO:1 and an antisense strand of SEQ ID NO:2;
sense strand of SEQ ID NO:3 and an antisense strand of SEQ ID NO:4;
sense strand of SEQ ID NO:5 and an antisense strand of SEQ ID NO:6;
sense strand of SEQ ID NO:7 and an antisense strand of SEQ ID NO:8;
sense strand of SEQ ID NO:9 and an antisense strand of SEQ ID NO:10;
sense strand of SEQ ID NO:11 and an antisense strand of SEQ ID NO:12;
sense strand of SEQ ID NO:13 and an antisense strand of SEQ ID NO:14;
sense strand of SEQ ID NO:15 and an antisense strand of SEQ ID NO:16;
sense strand of SEQ ID NO:17 and an antisense strand of SEQ ID NO:18;
sense strand of SEQ ID NO:19 and an antisense strand of SEQ ID NO:20;
sense strand of SEQ ID NO:21 and an antisense strand of SEQ ID NO:22;
sense strand of SEQ ID NO:23 and an antisense strand of SEQ ID NO:24;
or a sequence at least 90% identical thereto.

2. The siRNA molecule of claim 1, which is fully chemically-modified.

3. The siRNA molecule of claim 2, wherein each nucleotide in the siRNA molecule is modified with a 2′-O-methyl group or a 2′-fluoro group.

4. The siRNA molecule of claim 1, wherein the siRNA molecule comprises at least one phosphorothioate linkage.

5. The siRNA molecule of claim 4, wherein the siRNA molecule comprises one of the following pairs of sequences:
sense strand of SEQ ID NO:25 and an antisense strand of SEQ ID NO:26;
sense strand of SEQ ID NO:27 and an antisense strand of SEQ ID NO:28;
sense strand of SEQ ID NO:29 and an antisense strand of SEQ ID NO:30;
sense strand of SEQ ID NO:31 and an antisense strand of SEQ ID NO:32;
sense strand of SEQ ID NO:33 and an antisense strand of SEQ ID NO:34;
sense strand of SEQ ID NO:35 and an antisense strand of SEQ ID NO:36;
sense strand of SEQ ID NO:37 and an antisense strand of SEQ ID NO:38;
sense strand of SEQ ID NO:39 and an antisense strand of SEQ ID NO:40;
sense strand of SEQ ID NO:41 and an antisense strand of SEQ ID NO:42; or
sense strand of SEQ ID NO:43 and an antisense strand of SEQ ID NO:44.

6. A composition comprising the siRNA of claim 1 any one of claims 15.

7. A composition comprising two or more of the siRNA of claim 1 in any combination, wherein the two or more siRNAs each comprise a different sequence.

8. The composition of claim 6, further comprising a nanoparticle.

9. The composition of claim 8, wherein the nanoparticle is a nanoliposome.

10. A pharmaceutical composition comprising the siRNA of claim 1, and/or the composition of any one of claims 6-9 and a pharmaceutically acceptable carrier, optionally wherein the siRNA is conjugated to a ligand, antibody, or aptamer.

11. The pharmaceutical composition of claim 10, wherein the siRNA is conjugated to a ligand, antibody, or aptamer.

12. A method of inhibiting expression of a human IRF4 gene in a cell, the method comprising contacting the cell with the pharmaceutical composition of claim 10, thereby inhibiting expression of the human IRF4 gene in the cell.

13. A method of treating cancer in a subject in need thereof, wherein the cancer expresses a human IRF4 gene, the method comprising delivering to the subject the pharmaceutical composition of claim, thereby treating cancer in the subject.

14. The method of claims 13, wherein the cancer is multiple myeloma or any other IRF4 associated disease.

15. The method of claim 13, wherein the delivery is systemic delivery.

16. A pair of siRNA molecules comprising a c-Myc siRNA molecule targeted to a human c-Myc mRNA and an IRF4 siRNA molecule targeted to a human IRF4 mRNA, wherein the siRNA molecules comprise at least one chemical modification, and wherein the IRF4 siRNA molecule comprises one of the following pairs of sequences:
sense strand of SEQ ID NO:1 and an antisense strand of SEQ ID NO:2;
sense strand of SEQ ID NO:3 and an antisense strand of SEQ ID NO:4;
sense strand of SEQ ID NO:5 and an antisense strand of SEQ ID NO:6;
sense strand of SEQ ID NO:7 and an antisense strand of SEQ ID NO:8;
sense strand of SEQ ID NO:9 and an antisense strand of SEQ ID NO:10;
sense strand of SEQ ID NO:11 and an antisense strand of SEQ ID NO:12;
sense strand of SEQ ID NO:13 and an antisense strand of SEQ ID NO:14;
sense strand of SEQ ID NO:15 and an antisense strand of SEQ ID NO:16;
sense strand of SEQ ID NO:17 and an antisense strand of SEQ ID NO:18;
sense strand of SEQ ID NO:19 and an antisense strand of SEQ ID NO:20;
sense strand of SEQ ID NO:21 and an antisense strand of SEQ ID NO:22;
sense strand of SEQ ID NO:23 and an antisense strand of SEQ ID NO:24;
or a sequence at least 90% identical thereto.

17-28. (canceled)

29. A method of treating cancer in a subject in need thereof, wherein the cancer expresses a human IRF4 gene, the method comprising delivering to the subject the pair of siRNA molecules of claim 16, thereby treating cancer in the subject.

30-31. (canceled)

32. A siRNA multivalent chimeric molecule comprising:
an IRF4 siRNA molecule targeted to human IRF4 mRNA;
a c-Myc siRNA molecule targeted to human c-Myc mRNA; and
a phosphodiester linker region connecting the IRF4 siRNA and the c-Myc siRNA.

33-45. (canceled)

46. A method of treating cancer in a subject in need thereof, wherein the cancer expresses a human IRF4 gene, the method comprising delivering to the subject the siRNA of claim 32, thereby treating cancer in the subject.

47-48. (canceled)


SEQUENCE LISTING (91 sequences)

SEQ ID NO: 1 - RNA, 21 nt, synthetic construct
  source 1..21: mol_type = other RNA
  modified_base 20..21: mod_base = OTHER; note = Thymine
gggcactgtttaaaggaaatt

SEQ ID NO: 2 - RNA, 21 nt, synthetic construct
  source 1..21: mol_type = other RNA
  modified_base 20..21: mod_base = OTHER; note = Thymine
tttcctttaaacagtgccctt

SEQ ID NO: 3 - RNA, 21 nt, synthetic construct
  source 1..21: mol_type = other RNA
gaacaagagcaatgactttga

SEQ ID NO: 4 - RNA, 21 nt, synthetic construct
  source 1..21: mol_type = other RNA
aaagtcattgctcttgttcaa

SEQ ID NO: 5 - RNA, 23 nt, synthetic construct
  source 1..23: mol_type = other RNA
  modified_base 22..23: mod_base = OTHER; note = Thymine
aacaagagcaatgactttgagtt

SEQ ID NO: 6 - RNA, 23 nt, synthetic construct
  source 1..23: mol_type = other RNA
  modified_base 22..23: mod_base = OTHER; note = Thymine
ctcaaagtcattgctcttgtttt

SEQ ID NO: 7 - RNA, 21 nt, synthetic construct
  source 1..21: mol_type = other RNA
  modified_base 20..21: mod_base = OTHER; note = Thymine
gagcaatgactttgaggaatt

SEQ ID NO: 8 - RNA, 21 nt, synthetic construct
  source 1..21: mol_type = other RNA
  modified_base 20..21: mod_base = OTHER; note = Thymine
ttcctcaaagtcattgctctt

SEQ ID NO: 9 - RNA, 23 nt, synthetic construct
  source 1..23: mol_type = other RNA
  modified_base 22..23: mod_base = OTHER; note = Thymine
ggacatctcagacccgtacaatt

SEQ ID NO: 10 - RNA, 23 nt, synthetic construct
  source 1..23: mol_type = other RNA
  modified_base 22..23: mod_base = OTHER; note = Thymine
ttgtacgggtctgagatgtcctt

SEQ ID NO: 11 - RNA, 21 nt, synthetic construct
  source 1..21: mol_type = other RNA
  modified_base 20..21: mod_base = OTHER; note = Thymine
ccaacaaactggagagagatt

SEQ ID NO: 12 - RNA, 21 nt, synthetic construct
  source 1..21: mol_type = other RNA
  modified_base 20..21: mod_base = OTHER; note = Thymine
tctctctccagtttgttggtt

SEQ ID NO: 13 - RNA, 23 nt, synthetic construct
  source 1..23: mol_type = other RNA
  modified_base 22..23: mod_base = OTHER; note = Thymine
aagctctttgacacacagcagtt

SEQ ID NO: 14 - RNA, 23 nt, synthetic construct
  source 1..23: mol_type = other RNA
  modified_base 22..23: mod_base = OTHER; note = Thymine
ctgctgtgtgtcaaagagctttt

SEQ ID NO: 15 - RNA, 23 nt, synthetic construct
  source 1..23: mol_type = other RNA
  modified_base 22..23: mod_base = OTHER; note = Thymine
aagctcatcacagctcacgtatt

SEQ ID NO: 16 - RNA, 23 nt, synthetic construct
  source 1..23: mol_type = other RNA
  modified_base 22..23: mod_base = OTHER; note = Thymine
tacgtgagctgtgatgagctttt

SEQ ID NO: 17 - RNA, 27 nt, synthetic construct
  source 1..27: mol_type = other RNA
  modified_base 26..27: mod_base = OTHER; note = Thymine
cctttcagtattgacactgactagatt

SEQ ID NO: 18 - RNA, 27 nt, synthetic construct
  source 1..27: mol_type = other RNA
tctagtcagtgtcaatactgaaaggca

SEQ ID NO: 19 - RNA, 21 nt, synthetic construct
  source 1..21: mol_type = other RNA
  modified_base 20..21: mod_base = OTHER; note = Thymine
ctagtaatgtctaagtaattt

SEQ ID NO: 20 - RNA, 21 nt, synthetic construct
  source 1..21: mol_type = other RNA
attacttagacattactagat

SEQ ID NO: 21 - RNA, 23 nt, synthetic construct
  source 1..23: mol_type = other RNA
aagacctccacttgcttaagtgg

SEQ ID NO: 22 - RNA, 23 nt, synthetic construct
  source 1..23: mol_type = other RNA
  modified_base 22..23: mod_base = OTHER; note = Thymine
acttaagcaagtggaggtctttt

SEQ ID NO: 23 - RNA, 27 nt, synthetic construct
  source 1..27: mol_type = other RNA
  modified_base 26..27: mod_base = OTHER; note = Thymine
gataaagtggaattggcaaactagatt

SEQ ID NO: 24 - RNA, 27 nt, synthetic construct
  source 1..27: mol_type = other RNA
tctagtttgccaattccactttatcaa

SEQ ID NO: 25 - RNA, 21 nt, synthetic construct
  source 1..21: mol_type = other RNA
  modified_base 1: mod_base = OTHER; note = 2'-fluorouracil
  modified_base 2: mod_base = OTHER; note = 2'-O-methyluracil
  modified_base 3: mod_base = OTHER; note = 2'-fluoroguanine
  modified_base 1..3: mod_base = OTHER; note = Phosphorothioate bond
  modified_base 4: mod_base = OTHER; note = 2'-O-methyladenine
  modified_base 5: mod_base = OTHER; note = 2'-fluoroadenine
  modified_base 6: mod_base = OTHER; note = 2'-O-methylcytosine
  modified_base 7: mod_base = OTHER; note = 2'-fluoroadenine
  modified_base 8: mod_base = OTHER; note = 2'-O-methyladenine
  modified_base 9: mod_base = OTHER; note = 2'-fluoroguanine
  modified_base 10: mod_base = OTHER; note = 2'-fluoroadenine
  modified_base 11: mod_base = OTHER; note = 2'-fluoroguanine
  modified_base 12: mod_base = OTHER; note = 2'-O-methylcytosine
  modified_base 13: mod_base = OTHER; note = 2'-fluoroadenine
  modified_base 14: mod_base = OTHER; note = 2'-O-methyladenine
  modified_base 15: mod_base = OTHER; note = 2'-fluorouracil
  modified_base 16: mod_base = OTHER; note = 2'-O-methylguanine
  modified_base 17: mod_base = OTHER; note = 2'-fluoroadenine
  modified_base 18: mod_base = OTHER; note = 2'-O-methylcytosine
  modified_base 19: mod_base = OTHER; note = 2'-fluorouracil
  modified_base 20: mod_base = OTHER; note = 2'-O-methyluracil
  modified_base 21: mod_base = OTHER; note = 2'-fluorouracil
  modified_base 19..21: mod_base = OTHER; note = Phosphorothioate bond
ttgaacaagagcaatgacttt

SEQ ID NO: 26 - RNA, 23 nt, synthetic construct
  source 1..23: mol_type = other RNA
  modified_base 1: mod_base = OTHER; note = 2'-O-methyladenine
  modified_base 2: mod_base = OTHER; note = 2'-fluoroadenine
  modified_base 3: mod_base = OTHER; note = 2'-O-methyladenine
  modified_base 4: mod_base = OTHER; note = 2'-fluoroguanine
  modified_base 5: mod_base = OTHER; note = 2'-O-methyluracil
  modified_base 6: mod_base = OTHER; note = 2'-fluorocytosine
  modified_base 7: mod_base = OTHER; note = 2'-O-methyladenine
  modified_base 8: mod_base = OTHER; note = 2'-fluorouracil
  modified_base 9: mod_base = OTHER; note = 2'-O-methyluracil
  modified_base 10: mod_base = OTHER; note = 2'-fluoroguanine
  modified_base 11: mod_base = OTHER; note = 2'-O-methylcytosine
  modified_base 12: mod_base = OTHER; note = 2'-O-methyluracil
  modified_base 13: mod_base = OTHER; note = 2'-O-methylcytosine
  modified_base 14: mod_base = OTHER; note = 2'-fluorouracil
  modified_base 15: mod_base = OTHER; note = 2'-O-methyluracil
  modified_base 16: mod_base = OTHER; note = 2'-fluoroguanine
  modified_base 17: mod_base = OTHER; note = 2'-O-methyluracil
  modified_base 18: mod_base = OTHER; note = 2'-fluorouracil
  modified_base 19: mod_base = OTHER; note = 2'-O-methylcytosine
  modified_base 20: mod_base = OTHER; note = 2'-fluoroadenine
  modified_base 21: mod_base = OTHER; note = 2'-O-methyladenine
  modified_base 22: mod_base = OTHER; note = 2'-O-methyladenine
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
aaagtcattgctcttgttcaaag

SEQ ID NO: 27 - RNA, 21 nt, synthetic construct
  source 1..21: mol_type = other RNA
  modified_base 1: mod_base = OTHER; note = 2'-fluoroadenine
  modified_base 2: mod_base = OTHER; note = 2'-O-methyladenine
  modified_base 3: mod_base = OTHER; note = 2'-fluoroguanine
  modified_base 4: mod_base = OTHER; note = 2'-O-methyladenine
  modified_base 5: mod_base = OTHER; note = 2'-fluoroguanine
  modified_base 6: mod_base = OTHER; note = 2'-O-methylcytosine
  modified_base 7: mod_base = OTHER; note = 2'-fluoroadenine
  modified_base 8: mod_base = OTHER; note = 2'-O-methyladenine
  modified_base 9: mod_base = OTHER; note = 2'-fluorouracil
  modified_base 10: mod_base = OTHER; note = 2'-fluoroguanine
  modified_base 11: mod_base = OTHER; note = 2'-fluoroadenine
  modified_base 12: mod_base = OTHER; note = 2'-O-methylcytosine
  modified_base 13: mod_base = OTHER; note = 2'-fluorouracil
  modified_base 14: mod_base = OTHER; note = 2'-O-methyluracil
  modified_base 15: mod_base = OTHER; note = 2'-fluorouracil
  modified_base 16: mod_base = OTHER; note = 2'-O-methylguanine
  modified_base 17: mod_base = OTHER; note = 2'-fluoroadenine
  modified_base 18: mod_base = OTHER; note = 2'-O-methylguanine
  modified_base 19: mod_base = OTHER; note = 2'-fluoroguanine
  modified_base 20: mod_base = OTHER; note = 2'-O-methyladenine
  modified_base 21: mod_base = OTHER; note = 2'-fluoroadenine
  modified_base 1..3: mod_base = OTHER; note = Phosphorothioate bond
  modified_base 19..21: mod_base = OTHER; note = Phosphorothioate bond
aagagcaatgactttgaggaa

SEQ ID NO: 28 - RNA, 23 nt, synthetic construct
  source 1..23: mol_type = other RNA
  modified_base 1: mod_base = OTHER; note = 2'-O-methyluracil
  modified_base 2: mod_base = OTHER; note = 2'-fluorouracil
  modified_base 3: mod_base = OTHER; note = 2'-O-methylcytosine
  modified_base 4: mod_base = OTHER; note = 2'-fluorocytosine
  modified_base 5: mod_base = OTHER; note = 2'-O-methyluracil
  modified_base 6: mod_base = OTHER; note = 2'-fluorocytosine
  modified_base 7: mod_base = OTHER; note = 2'-O-methyladenine
  modified_base 8: mod_base = OTHER; note = 2'-fluoroadenine
  modified_base 9: mod_base = OTHER; note = 2'-O-methyladenine
  modified_base 10: mod_base = OTHER; note = 2'-fluoroguanine
  modified_base 11: mod_base = OTHER; note = 2'-O-methyluracil
  modified_base 12: mod_base = OTHER; note = 2'-O-methylcytosine
  modified_base 13: mod_base = OTHER; note = 2'-O-methyladenine
  modified_base 14: mod_base = OTHER; note = 2'-fluorouracil
  modified_base 15: mod_base = OTHER; note = 2'-O-methyluracil
  modified_base 16: mod_base = OTHER; note = 2'-fluoroguanine
  modified_base 17: mod_base = OTHER; note = 2'-O-methylcytosine
  modified_base 18: mod_base = OTHER; note = 2'-fluorouracil
  modified_base 19: mod_base = OTHER; note = 2'-O-methylcytosine
  modified_base 20: mod_base = OTHER; note = 2'-fluorouracil
  modified_base 21: mod_base = OTHER; note = 2'-O-methyluracil
  modified_base 22: mod_base = OTHER; note = 2'-O-methylguanine
  modified_base 23: mod_base = OTHER; note = 2'-O-methyluracil
  modified_base 1..3: mod_base = OTHER; note = Phosphorothioate bond
  modified_base 21..23: mod_base = OTHER; note = Phosphorothioate bond
ttcctcaaagtcattgctcttgt

SEQ ID NO: 29 - RNA, 21 nt, synthetic construct
  source 1..21: mol_type = other RNA
  modified_base 1: mod_base = OTHER; note = 2'-fluoroguanine
  modified_base 2: mod_base = OTHER; note = 2'-O-methylguanine
  modified_base 3: mod_base = OTHER; note = 2'-fluoroadenine
  modified_base 4: mod_base = OTHER; note = 2'-O-methylcytosine
  modified_base 5: mod_base = OTHER; note = 2'-fluoroadenine
  modified_base 6: mod_base = OTHER; note = 2'-O-methyluracil
  modified_base 7: mod_base = OTHER; note = 2'-fluorocytosine
  modified_base 8: mod_base = OTHER; note = 2'-O-methyluracil
  modified_base 9: mod_base = OTHER; note = 2'-fluorocytosine
  modified_base 10: mod_base = OTHER; note = 2'-fluoroadenine
  modified_base 11: mod_base = OTHER; note = 2'-fluoroguanine
  modified_base 12: mod_base = OTHER; note = 2'-O-methyladenine
  modified_base 13: mod_base = OTHER; note = 2'-fluorocytosine
  modified_base 14: mod_base = OTHER; note = 2'-O-methylcytosine
  modified_base 15: mod_base = OTHER; note = 2'-fluorocytosine
  modified_base 16: mod_base = OTHER; note = 2'-O-methylguanine
  modified_base 17: mod_base = OTHER; note = 2'-fluorouracil
  modified_base 18: mod_base = OTHER; note = 2'-O-methyladenine
  modified_base 19: mod_base = OTHER; note = 2'-fluorocytosine
  modified_base 20: mod_base = OTHER; note = 2'-O-methyladenine
  modified_base 21: mod_base = OTHER; note = 2'-fluoroadenine
  modified_base 1..3: mod_base = OTHER; note = Phosphorothioate bond
  modified_base 19..21: mod_base = OTHER; note = Phosphorothioate bond
ggacatctcagacccgtacaa

SEQ ID NO: 30 - RNA, 23 nt, synthetic construct
  source 1..23: mol_type = other RNA
  modified_base 1: mod_base = OTHER; note = 2'-O-methyluracil
  modified_base 2: mod_base = OTHER; note = 2'-fluorouracil
  modified_base 3: mod_base = OTHER; note = 2'-O-methylguanine
  modified_base 4: mod_base = OTHER; note = 2'-fluorouracil
  modified_base 5: mod_base = OTHER; note = 2'-O-methyladenine
  modified_base 6: mod_base = OTHER; note = 2'-fluorocytosine
  modified_base 7: mod_base = OTHER; note = 2'-O-methylguanine
  modified_base 8: mod_base = OTHER; note = 2'-fluoroguanine
  modified_base 9: mod_base = OTHER; note = 2'-O-methylguanine
  modified_base 10: mod_base = OTHER; note = 2'-fluorouracil
  modified_base 11: mod_base = OTHER; note = 2'-O-methylcytosine
  modified_base 12: mod_base = OTHER; note = 2'-O-methyluracil
  modified_base 13: mod_base = OTHER; note = 2'-O-methylguanine
  modified_base 14: mod_base = OTHER; note = 2'-fluoroadenine
  modified_base 15: mod_base = OTHER; note = 2'-O-methylguanine
  modified_base 16: mod_base = OTHER; note = 2'-fluoroadenine
  modified_base 17: mod_base = OTHER; note = 2'-O-methyluracil
  modified_base 18: mod_base = OTHER; note = 2'-fluoroguanine
  modified_base 19: mod_base = OTHER; note = 2'-O-methyluracil
  modified_base 20: mod_base = OTHER; note = 2'-fluorocytosine
  modified_base 21: mod_base = OTHER; note = 2'-O-methylcytosine
  modified_base 22: mod_base = OTHER; note = 2'-O-methyladenine
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
ttgtacgggtctgagatgtccag

SEQ ID NO: 31 - RNA, 21 nt, synthetic construct
  source 1..21: mol_type = other RNA
  modified_base 1: mod_base = OTHER; note = 2'-O-methylguanine
  modified_base 2: mod_base = OTHER; note = 2'-O-methylcytosine
  modified_base 3: mod_base = OTHER; note = 2'-fluorocytosine
  modified_base 4: mod_base = OTHER; note = 2'-O-methylcytosine
  modified_base 5: mod_base = OTHER; note = 2'-fluoroadenine
  modified_base 6: mod_base = OTHER; note = 2'-O-methyladenine
  modified_base 7: mod_base = OTHER; note = 2'-fluorocytosine
  modified_base 8: mod_base = OTHER; note = 2'-O-methyladenine
  modified_base 9..10: mod_base = OTHER; note = 2'-fluoroadenine
  modified_base 11: mod_base = OTHER; note = 2'-fluorocytosine
  modified_base 12: mod_base = OTHER; note = 2'-O-methyluracil
  modified_base 13: mod_base = OTHER; note = 2'-fluoroguanine
  modified_base 14: mod_base = OTHER; note = 2'-O-methylguanine
  modified_base 15: mod_base = OTHER; note = 2'-fluoroadenine
  modified_base 16: mod_base = OTHER; note = 2'-O-methylguanine
  modified_base 17: mod_base = OTHER; note = 2'-fluoroadenine
  modified_base 18: mod_base = OTHER; note = 2'-O-methylguanine
  modified_base 19: mod_base = OTHER; note = 2'-fluoroadenine
  modified_base 20: mod_base = OTHER; note = 2'-O-methylguanine
  modified_base 21: mod_base = OTHER; note = 2'-fluoroadenine
  modified_base 1..3: mod_base = OTHER; note = Phosphorothioate bond
  modified_base 19..21: mod_base = OTHER; note = Phosphorothioate bond
gcccaacaaactggagagaga

SEQ ID NO: 32 - RNA, 23 nt, synthetic construct
  source 1..23: mol_type = other RNA
  modified_base 1: mod_base = OTHER; note = 2'-O-methyluracil
  modified_base 2: mod_base = OTHER; note = 2'-fluorocytosine
  modified_base 3: mod_base = OTHER; note = 2'-O-methyluracil
  modified_base 4: mod_base = OTHER; note = 2'-fluorocytosine
  modified_base 5: mod_base = OTHER; note = 2'-O-methyluracil
  modified_base 6: mod_base = OTHER; note = 2'-fluorocytosine
  modified_base 7: mod_base = OTHER; note = 2'-O-methyluracil
  modified_base 8: mod_base = OTHER; note = 2'-fluorocytosine
  modified_base 9: mod_base = OTHER; note = 2'-O-methylcytosine
  modified_base 10: mod_base = OTHER; note = 2'-fluoroadenine
  modified_base 11: mod_base = OTHER; note = 2'-O-methylguanine
  modified_base 12..13: mod_base = OTHER; note = 2'-O-methyluracil
  modified_base 14: mod_base = OTHER; note = 2'-fluorouracil
  modified_base 15: mod_base = OTHER; note = 2'-O-methylguanine
  modified_base 16: mod_base = OTHER; note = 2'-fluorouracil
  modified_base 17: mod_base = OTHER; note = 2'-O-methyluracil
  modified_base 18: mod_base = OTHER; note = 2'-fluoroguanine
  modified_base 19: mod_base = OTHER; note = 2'-O-methylguanine
  modified_base 20: mod_base = OTHER; note = 2'-fluoroguanine
  modified_base 21..22: mod_base = OTHER; note = 2'-O-methylcytosine
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
tctctctccagtttgttgggccg

SEQ ID NO: 33 - RNA, 21 nt, synthetic construct
  source 1..21: mol_type = other RNA
  modified_base 1: mod_base = OTHER; note = 2'-fluoroadenine
  modified_base 2: mod_base = OTHER; note = 2'-O-methyladenine
  modified_base 3: mod_base = OTHER; note = 2'-fluoroguanine
  modified_base 4: mod_base = OTHER; note = 2'-O-methylcytosine
  modified_base 5: mod_base = OTHER; note = 2'-fluorouracil
  modified_base 6: mod_base = OTHER; note = 2'-O-methylcytosine
  modified_base 7: mod_base = OTHER; note = 2'-fluoroadenine
  modified_base 8: mod_base = OTHER; note = 2'-O-methyluracil
  modified_base 9: mod_base = OTHER; note = 2'-fluorocytosine
  modified_base 10: mod_base = OTHER; note = 2'-fluoroadenine
  modified_base 11: mod_base = OTHER; note = 2'-fluorocytosine
  modified_base 12: mod_base = OTHER; note = 2'-O-methyladenine
  modified_base 13: mod_base = OTHER; note = 2'-fluoroguanine
  modified_base 14: mod_base = OTHER; note = 2'-O-methylcytosine
  modified_base 15: mod_base = OTHER; note = 2'-fluorouracil
  modified_base 16: mod_base = OTHER; note = 2'-O-methylcytosine
  modified_base 17: mod_base = OTHER; note = 2'-fluoroadenine
  modified_base 18: mod_base = OTHER; note = 2'-O-methylcytosine
  modified_base 19: mod_base = OTHER; note = 2'-fluoroguanine
  modified_base 20: mod_base = OTHER; note = 2'-O-methyluracil
  modified_base 21: mod_base = OTHER; note = 2'-fluoroadenine
  modified_base 1..3: mod_base = OTHER; note = Phosphorothioate bond
  modified_base 19..21: mod_base = OTHER; note = Phosphorothioate bond
aagctcatcacagctcacgta

SEQ ID NO: 34 - RNA, 23 nt, synthetic construct
  source 1..23: mol_type = other RNA
  modified_base 1: mod_base = OTHER; note = 2'-O-methyluracil
  modified_base 2: mod_base = OTHER; note = 2'-fluoroadenine
  modified_base 3: mod_base = OTHER; note = 2'-O-methylcytosine
  modified_base 4: mod_base = OTHER; note = 2'-fluoroguanine
  modified_base 5: mod_base = OTHER; note = 2'-O-methyluracil
  modified_base 6: mod_base = OTHER; note = 2'-fluoroguanine
  modified_base 7: mod_base = OTHER; note = 2'-O-methyladenine
  modified_base 8: mod_base = OTHER; note = 2'-fluoroguanine
  modified_base 9: mod_base = OTHER; note = 2'-O-methylcytosine
  modified_base 10: mod_base = OTHER; note = 2'-fluorouracil
  modified_base 11: mod_base = OTHER; note = 2'-O-methylguanine
  modified_base 12: mod_base = OTHER; note = 2'-O-methyluracil
  modified_base 13: mod_base = OTHER; note = 2'-O-methylguanine
  modified_base 14: mod_base = OTHER; note = 2'-fluoroadenine
  modified_base 15: mod_base = OTHER; note = 2'-O-methyluracil
  modified_base 16: mod_base = OTHER; note = 2'-fluoroguanine
  modified_base 17: mod_base = OTHER; note = 2'-O-methyladenine
  modified_base 18: mod_base = OTHER; note = 2'-fluoroguanine
  modified_base 19: mod_base = OTHER; note = 2'-O-methylcytosine
  modified_base 20: mod_base = OTHER; note = 2'-fluorouracil
  modified_base 21..22: mod_base = OTHER; note = 2'-O-methyluracil
  modified_base 23: mod_base = OTHER; note = 2'-O-methylcytosine
  modified_base 1..3: mod_base = OTHER; note = Phosphorothioate bond
  modified_base 21..23: mod_base = OTHER; note = Phosphorothioate bond
tacgtgagctgtgatgagctttc

SEQ ID NO: 35 - RNA, 21 nt, synthetic construct
  source 1..21: mol_type = other RNA
  modified_base 1: mod_base = OTHER; note = 2'-fluoroadenine
  modified_base 2: mod_base = OTHER; note = 2'-O-methyluracil
  modified_base 3: mod_base = OTHER; note = 2'-fluorocytosine
  modified_base 4: mod_base = OTHER; note = 2'-O-methyluracil
  modified_base 5: mod_base = OTHER; note = 2'-fluoroadenine
  modified_base 6: mod_base = OTHER; note = 2'-O-methylguanine
  modified_base 7: mod_base = OTHER; note = 2'-fluorouracil
  modified_base 8: mod_base = OTHER; note = 2'-O-methyladenine
  modified_base 9: mod_base = OTHER; note = 2'-fluoroadenine
  modified_base 10: mod_base = OTHER; note = 2'-fluorouracil
  modified_base 11: mod_base = OTHER; note = 2'-fluoroguanine
  modified_base 12: mod_base = OTHER; note = 2'-O-methyluracil
  modified_base 13: mod_base = OTHER; note = 2'-fluorocytosine
  modified_base 14: mod_base = OTHER; note = 2'-O-methyluracil
  modified_base 15: mod_base = OTHER; note = 2'-fluoroadenine
  modified_base 16: mod_base = OTHER; note = 2'-O-methyladenine
  modified_base 17: mod_base = OTHER; note = 2'-fluoroguanine
  modified_base 18: mod_base = OTHER; note = 2'-O-methyluracil
  modified_base 19: mod_base = OTHER; note = 2'-fluoroadenine
  modified_base 20: mod_base = OTHER; note = 2'-O-methyladenine
  modified_base 21: mod_base = OTHER; note = 2'-fluorouracil
  modified_base 1..3: mod_base = OTHER; note = Phosphorothioate bond
  modified_base 19..21: mod_base = OTHER; note = Phosphorothioate bond
atctagtaatgtctaagtaat

SEQ ID NO: 36 - RNA, 23 nt, synthetic construct
  source 1..23: mol_type = other RNA
  modified_base 1: mod_base = OTHER; note = 2'-O-methyladenine
  modified_base 2: mod_base = OTHER; note = 2'-fluorouracil
  modified_base 3: mod_base = OTHER; note = 2'-O-methyluracil
  modified_base 4: mod_base = OTHER; note = 2'-fluoroadenine
  modified_base 5: mod_base = OTHER; note = 2'-O-methylcytosine
  modified_base 6: mod_base = OTHER; note = 2'-fluorouracil
  modified_base 7: mod_base = OTHER; note = 2'-O-methyluracil
  modified_base 8: mod_base = OTHER; note = 2'-fluoroadenine
  modified_base 9: mod_base = OTHER; note = 2'-O-methylguanine
  modified_base 10: mod_base = OTHER; note = 2'-fluoroadenine
  modified_base 11: mod_base = OTHER; note = 2'-O-methylcytosine
  modified_base 12: mod_base = OTHER; note = 2'-O-methyladenine
  modified_base 13: mod_base = OTHER; note = 2'-O-methyluracil
  modified_base 14: mod_base = OTHER; note = 2'-fluorouracil
  modified_base 15: mod_base = OTHER; note = 2'-O-methyladenine
  modified_base 16: mod_base = OTHER; note = 2'-fluorocytosine
  modified_base 17: mod_base = OTHER; note = 2'-O-methyluracil
  modified_base 18: mod_base = OTHER; note = 2'-fluoroadenine
  modified_base 19: mod_base = OTHER; note = 2'-O-methylguanine
  modified_base 20: mod_base = OTHER; note = 2'-fluoroadenine
  modified_base 21..22: mod_base = OTHER; note = 2'-O-methyluracil
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
attacttagacattactagattg

SEQ ID NO: 37 - RNA, 21 nt, synthetic construct
  source 1..21: mol_type = other RNA
  modified_base 1: mod_base = OTHER; note = 2'-fluoroadenine
  modified_base 2: mod_base = OTHER; note = 2'-O-methyladenine
  modified_base 3: mod_base = OTHER; note = 2'-fluoroguanine
  modified_base 4: mod_base = OTHER; note = 2'-O-methyladenine
  modified_base 5: mod_base = OTHER; note = 2'-fluorocytosine
  modified_base 6: mod_base = OTHER; note = 2'-O-methylcytosine
  modified_base 7: mod_base = OTHER; note = 2'-fluorouracil
  modified_base 8: mod_base = OTHER; note = 2'-O-methylcytosine
  modified_base 9: mod_base = OTHER; note = 2'-fluorocytosine
  modified_base 10: mod_base = OTHER; note = 2'-fluoroadenine
  modified_base 11: mod_base = OTHER; note = 2'-fluorocytosine
  modified_base 12: mod_base = OTHER; note = 2'-O-methyluracil
  modified_base 13: mod_base = OTHER; note = 2'-fluorouracil
  modified_base 14: mod_base = OTHER; note = 2'-O-methylguanine
  modified_base 15: mod_base = OTHER; note = 2'-fluorocytosine
  modified_base 16: mod_base = OTHER; note = 2'-O-methyluracil
  modified_base 17: mod_base = OTHER; note = 2'-fluorouracil
  modified_base 18: mod_base = OTHER; note = 2'-O-methyladenine
  modified_base 19: mod_base = OTHER; note = 2'-fluoroadenine
  modified_base 20: mod_base = OTHER; note = 2'-O-methylguanine
  modified_base 21: mod_base = OTHER; note = 2'-fluorouracil
  modified_base 1..3: mod_base = OTHER; note = Phosphorothioate bond
  modified_base 19..21: mod_base = OTHER; note = Phosphorothioate bond
aagacctccacttgcttaagt

SEQ ID NO: 38 - RNA, 23 nt, synthetic construct
  source 1..23: mol_type = other RNA
  modified_base 1: mod_base = OTHER; note = 2'-O-methyladenine
  modified_base 2: mod_base = OTHER; note = 2'-fluorocytosine
  modified_base 3: mod_base = OTHER; note = 2'-O-methyluracil
  modified_base 4: mod_base = OTHER; note = 2'-fluorouracil
  modified_base 5: mod_base = OTHER; note = 2'-O-methyladenine
  modified_base 6: mod_base = OTHER; note = 2'-fluoroadenine
  modified_base 7: mod_base = OTHER; note = 2'-O-methylguanine
  modified_base 8: mod_base = OTHER; note = 2'-fluorocytosine
  modified_base 9: mod_base = OTHER; note = 2'-O-methyladenine
  modified_base 10: mod_base = OTHER; note = 2'-fluoroadenine
  modified_base 11: mod_base = OTHER; note = 2'-O-methylguanine
  modified_base 12: mod_base = OTHER; note = 2'-O-methyluracil
  modified_base 13: mod_base = OTHER; note = 2'-O-methylguanine
  modified_base 14: mod_base = OTHER; note = 2'-fluoroguanine
  modified_base 15: mod_base = OTHER; note = 2'-O-methyladenine
  modified_base 16: mod_base = OTHER; note = 2'-fluoroguanine
  modified_base 17: mod_base = OTHER; note = 2'-O-methylguanine
  modified_base 18: mod_base = OTHER; note = 2'-fluorouracil
  modified_base 19: mod_base = OTHER; note = 2'-O-methylcytosine
  modified_base 20: mod_base = OTHER; note = 2'-fluorouracil
  modified_base 21..22: mod_base = OTHER; note = 2'-O-methyluracil
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
acttaagcaagtggaggtctttg

SEQ ID NO: 39 - RNA, 21 nt, synthetic construct
  source 1..21: mol_type = other RNA
  modified_base 1: mod_base = OTHER; note = 2'-O-methylguanine
  modified_base 2..4: mod_base = OTHER; note = 2'-O-methylcytosine
  modified_base 5..6: mod_base = OTHER; note = 2'-O-methyladenine
  modified_base 7: mod_base = OTHER; note = 2'-fluorocytosine
  modified_base 8: mod_base = OTHER; note = 2'-O-methyladenine
  modified_base 9..10: mod_base = OTHER; note = 2'-fluoroadenine
  modified_base 11: mod_base = OTHER; note = 2'-fluorocytosine
  modified_base 12: mod_base = OTHER; note = 2'-O-methyluracil
  modified_base 13..14: mod_base = OTHER; note = 2'-O-methylguanine
  modified_base 15: mod_base = OTHER; note = 2'-O-methyladenine
  modified_base 16: mod_base = OTHER; note = 2'-O-methylguanine
  modified_base 17: mod_base = OTHER; note = 2'-O-methyladenine
  modified_base 18: mod_base = OTHER; note = 2'-O-methylguanine
  modified_base 19: mod_base = OTHER; note = 2'-O-methyladenine
  modified_base 20: mod_base = OTHER; note = 2'-O-methylguanine
  modified_base 21: mod_base = OTHER; note = 2'-O-methyladenine
  modified_base 1..3: mod_base = OTHER; note = Phosphorothioate bond
  modified_base 19..21: mod_base = OTHER; note = Phosphorothioate bond
gcccaacaaactggagagaga

SEQ ID NO: 40 - RNA, 23 nt, synthetic construct
  source 1..23: mol_type = other RNA
  modified_base 1: mod_base = OTHER; note = 2'-O-methyluracil
  modified_base 2: mod_base = OTHER; note = 2'-fluorocytosine
  modified_base 3: mod_base = OTHER; note = 2'-O-methyluracil
  modified_base 4: mod_base = OTHER; note = 2'-O-methylcytosine
  modified_base 5: mod_base = OTHER; note = 2'-O-methyluracil
  modified_base 6: mod_base = OTHER; note = 2'-fluorocytosine
  modified_base 7: mod_base = OTHER; note = 2'-O-methyluracil
  modified_base 8..9: mod_base = OTHER; note = 2'-O-methylcytosine
  modified_base 10: mod_base = OTHER; note = 2'-O-methyladenine
  modified_base 11: mod_base = OTHER; note = 2'-O-methylguanine
  modified_base 12..13: mod_base = OTHER; note = 2'-O-methyluracil
  modified_base 14: mod_base = OTHER; note = 2'-fluorouracil
  modified_base 15: mod_base = OTHER; note = 2'-O-methylguanine
  modified_base 16: mod_base = OTHER; note = 2'-fluorouracil
  modified_base 17: mod_base = OTHER; note = 2'-O-methyluracil
  modified_base 18..20: mod_base = OTHER; note = 2'-O-methylguanine
  modified_base 21..22: mod_base = OTHER; note = 2'-O-methylcytosine
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
tctctctccagtttgttgggccg

SEQ ID NO: 41 - RNA, 21 nt, synthetic construct
  source 1..21: mol_type = other RNA
  modified_base 1: mod_base = OTHER; note = 2'-O-methyladenine
  modified_base 2: mod_base = OTHER; note = 2'-O-methyluracil
  modified_base 3: mod_base = OTHER; note = 2'-O-methylcytosine
  modified_base 4: mod_base = OTHER; note = 2'-O-methyluracil
  modified_base 5: mod_base = OTHER; note = 2'-O-methyladenine
  modified_base 6: mod_base = OTHER; note = 2'-O-methylguanine
  modified_base 7: mod_base = OTHER; note = 2'-fluorouracil
  modified_base 8: mod_base = OTHER; note = 2'-O-methyladenine
  modified_base 9: mod_base = OTHER; note = 2'-fluoroadenine
  modified_base 10: mod_base = OTHER; note = 2'-fluorouracil
  modified_base 11: mod_base = OTHER; note = 2'-fluoroguanine
  modified_base 12: mod_base = OTHER; note = 2'-O-methyluracil
  modified_base 13: mod_base = OTHER; note = 2'-O-methylcytosine
  modified_base 14: mod_base = OTHER; note = 2'-O-methyluracil
  modified_base 15..16: mod_base = OTHER; note = 2'-O-methyladenine
  modified_base 17: mod_base = OTHER; note = 2'-O-methylguanine
  modified_base 18: mod_base = OTHER; note = 2'-O-methyluracil
  modified_base 19..20: mod_base = OTHER; note = 2'-O-methyladenine
  modified_base 21: mod_base = OTHER; note = 2'-O-methyluracil
  modified_base 1..3: mod_base = OTHER; note = Phosphorothioate bond
  modified_base 19..21: mod_base = OTHER; note = Phosphorothioate bond
atctagtaatgtctaagtaat

SEQ ID NO: 42 - RNA, 23 nt, synthetic construct
  source 1..23: mol_type = other RNA
  modified_base 1: mod_base = OTHER; note = 2'-O-methyladenine
  modified_base 2: mod_base = OTHER; note = 2'-fluorouracil
  modified_base 3: mod_base = OTHER; note = 2'-O-methyluracil
  modified_base 4: mod_base = OTHER; note = 2'-O-methyladenine
  modified_base 5: mod_base = OTHER; note = 2'-O-methylcytosine
  modified_base 6: mod_base = OTHER; note = 2'-fluorouracil
  modified_base 7: mod_base = OTHER; note = 2'-O-methyluracil
  modified_base 8: mod_base = OTHER; note = 2'-O-methyladenine
  modified_base 9: mod_base = OTHER; note = 2'-O-methylguanine
  modified_base 10: mod_base = OTHER; note = 2'-O-methyladenine
  modified_base 11: mod_base = OTHER; note = 2'-O-methylcytosine
  modified_base 12: mod_base = OTHER; note = 2'-O-methyladenine
  modified_base 13: mod_base = OTHER; note = 2'-O-methyluracil
  modified_base 14: mod_base = OTHER; note = 2'-fluorouracil
  modified_base 15: mod_base = OTHER; note = 2'-O-methyladenine
  modified_base 16: mod_base = OTHER; note = 2'-fluorocytosine
  modified_base 17: mod_base = OTHER; note = 2'-O-methyluracil
  modified_base 18: mod_base = OTHER; note = 2'-O-methyladenine
  modified_base 19: mod_base = OTHER; note = 2'-O-methylguanine
  modified_base 20: mod_base = OTHER; note = 2'-O-methyladenine
  modified_base 21..22: mod_base = OTHER; note = 2'-O-methyluracil
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
attacttagacattactagattg

SEQ ID NO: 43 - RNA, 21 nt, synthetic construct
  source 1..21: mol_type = other RNA
  modified_base 1..2: mod_base = OTHER; note = 2'-O-methyladenine
  modified_base 3: mod_base = OTHER; note = 2'-O-methylguanine
  modified_base 4: mod_base = OTHER; note = 2'-O-methyladenine
  modified_base 5..6: mod_base = OTHER; note = 2'-O-methylcytosine
  modified_base 7: mod_base = OTHER; note = 2'-fluorouracil
  modified_base 8: mod_base = OTHER; note = 2'-O-methylcytosine
  modified_base 9: mod_base = OTHER; note = 2'-fluorocytosine
  modified_base 10: mod_base = OTHER; note = 2'-fluoroadenine
  modified_base 11: mod_base = OTHER; note = 2'-fluorocytosine
  modified_base 12..13: mod_base = OTHER; note = 2'-O-methyluracil
  modified_base 14: mod_base = OTHER; note = 2'-O-methylguanine
  modified_base 15: mod_base = OTHER; note = 2'-O-methylcytosine
  modified_base 16..17: mod_base = OTHER; note = 2'-O-methyluracil
  modified_base 18..19: mod_base = OTHER; note = 2'-O-methyladenine
  modified_base 20: mod_base = OTHER; note = 2'-O-methylguanine
  modified_base 21: mod_base = OTHER; note = 2'-O-methyluracil
  modified_base 1..3: mod_base = OTHER; note = Phosphorothioate bond
  modified_base 19..21: mod_base = OTHER; note = Phosphorothioate bond
aagacctccacttgcttaagt

SEQ ID NO: 44 - RNA, 23 nt, synthetic construct
  source 1..23: mol_type = other RNA
  modified_base 1: mod_base = OTHER; note = 2'-O-methyladenine
  modified_base 2: mod_base = OTHER; note = 2'-fluorocytosine
  modified_base 3..4: mod_base = OTHER; note = 2'-O-methyluracil
  modified_base 5: mod_base = OTHER; note = 2'-O-methyladenine
  modified_base 6: mod_base = OTHER; note = 2'-fluoroadenine
  modified_base 7: mod_base = OTHER; note = 2'-O-methylguanine
  modified_base 8: mod_base = OTHER; note = 2'-O-methylcytosine
  modified_base 9..10: mod_base = OTHER; note = 2'-O-methyladenine
  modified_base 11: mod_base = OTHER; note = 2'-O-methylguanine
  modified_base 12: mod_base = OTHER; note = 2'-O-methyluracil
  modified_base 13: mod_base = OTHER; note = 2'-O-methylguanine
  modified_base 14: mod_base = OTHER; note = 2'-fluoroguanine
  modified_base 15: mod_base = OTHER; note = 2'-O-methyladenine
  modified_base 16: mod_base = OTHER; note = 2'-fluoroguanine
  modified_base 17: mod_base = OTHER; note = 2'-O-methylguanine
  modified_base 18: mod_base = OTHER; note = 2'-O-methyluracil
  modified_base 19: mod_base = OTHER; note = 2'-O-methylcytosine
  modified_base 20..22: mod_base = OTHER; note = 2'-O-methyluracil
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
acttaagcaagtggaggtctttg

SEQ ID NO: 45 - RNA, 48 nt, synthetic construct
  source 1..48: mol_type = other RNA
  modified_base 1: mod_base = OTHER; note = 2'-O-methyladenine
  modified_base 2: mod_base = OTHER; note = 2'-O-methylcytosine
  modified_base 3: mod_base = OTHER; note = 2'-O-methylguanine
  modified_base 4: mod_base = OTHER; note = 2'-O-methyladenine
  modified_base 5: mod_base = OTHER; note = 2'-O-methylguanine
  modified_base 6: mod_base = OTHER; note = 2'-O-methyladenine
  modified_base 7: mod_base = OTHER; note = 2'-fluoroadenine
  modified_base 8: mod_base = OTHER; note = 2'-O-methylcytosine
  modified_base 9: mod_base = OTHER; note = 2'-fluoroadenine
  modified_base 10: mod_base = OTHER; note = 2'-fluoroguanine
  modified_base 11: mod_base = OTHER; note = 2'-fluorouracil
  modified_base 12: mod_base = OTHER; note = 2'-O-methyluracil
  modified_base 13: mod_base = OTHER; note = 2'-O-methylguanine
  modified_base 14..16: mod_base = OTHER; note = 2'-O-methyladenine
  modified_base 17: mod_base = OTHER; note = 2'-O-methylcytosine
  modified_base 18: mod_base = OTHER; note = 2'-O-methyladenine
  modified_base 19: mod_base = OTHER; note = 2'-O-methylcytosine
  modified_base 20..21: mod_base = OTHER; note = 2'-O-methyladenine
  modified_base 22..25: mod_base = OTHER; note = Thymine
  modified_base 26: mod_base = OTHER; note = 2'-O-methyladenine
  modified_base 27: mod_base = OTHER; note = 2'-fluorouracil
  modified_base 28: mod_base = OTHER; note = 2'-O-methyluracil
  modified_base 29: mod_base = OTHER; note = 2'-O-methyladenine
  modified_base 30: mod_base = OTHER; note = 2'-O-methylcytosine
  modified_base 31: mod_base = OTHER; note = 2'-fluorouracil
  modified_base 32: mod_base = OTHER; note = 2'-O-methyluracil
  modified_base 33: mod_base = OTHER; note = 2'-O-methyladenine
  modified_base 34: mod_base = OTHER; note = 2'-O-methylguanine
  modified_base 35: mod_base = OTHER; note = 2'-O-methyladenine
  modified_base 36: mod_base = OTHER; note = 2'-O-methylcytosine
  modified_base 37: mod_base = OTHER; note = 2'-O-methyladenine
  modified_base 38: mod_base = OTHER; note = 2'-O-methyluracil
  modified_base 39: mod_base = OTHER; note = 2'-fluorouracil
  modified_base 40: mod_base = OTHER; note = 2'-O-methyladenine
  modified_base 41: mod_base = OTHER; note = 2'-fluorocytosine
  modified_base 42: mod_base = OTHER; note = 2'-O-methyluracil
  modified_base 43: mod_base = OTHER; note = 2'-O-methyladenine
  modified_base 44: mod_base = OTHER; note = 2'-O-methylguanine
  modified_base 45: mod_base = OTHER; note = 2'-O-methyladenine
  modified_base 46..47: mod_base = OTHER; note = 2'-O-methyluracil
  modified_base 48: mod_base = OTHER; note = 2'-O-methylguanine
  modified_base 1..3: mod_base = OTHER; note = Phosphorothioate bond
  modified_base 19..21: mod_base = OTHER; note = Phosphorothioate bond
  modified_base 26..28: mod_base = OTHER; note = Phosphorothioate bond
  modified_base 46..48: mod_base = OTHER; note = Phosphorothioate bond
acgagaacagttgaaacacaattttattacttagacattactagattg

SEQ ID NO: 46 - RNA, 23 nt, synthetic construct
  source 1..23: mol_type = other RNA
  modified_base 1: mod_base = OTHER; note = 2'-O-methyluracil
  modified_base 2: mod_base = OTHER; note = 2'-fluorouracil
  modified_base 3: mod_base = OTHER; note = 2'-O-methylguanine
  modified_base 4: mod_base = OTHER; note = 2'-O-methyluracil
  modified_base 5: mod_base = OTHER; note = 2'-O-methylguanine
  modified_base 6: mod_base = OTHER; note = 2'-fluorouracil
  modified_base 7..8: mod_base = OTHER; note = 2'-O-methyluracil
  modified_base 9: mod_base = OTHER; note = 2'-O-methylcytosine
  modified_base 10..11: mod_base = OTHER; note = 2'-O-methyladenine
  modified_base 12: mod_base = OTHER; note = 2'-O-methylcytosine
  modified_base 13: mod_base = OTHER; note = 2'-O-methyluracil
  modified_base 14: mod_base = OTHER; note = 2'-fluoroguanine
  modified_base 15: mod_base = OTHER; note = 2'-O-methyluracil
  modified_base 16: mod_base = OTHER; note = 2'-fluorouracil
  modified_base 17: mod_base = OTHER; note = 2'-O-methylcytosine
  modified_base 18: mod_base = OTHER; note = 2'-O-methyluracil
  modified_base 19: mod_base = OTHER; note = 2'-O-methylcytosine
  modified_base 20: mod_base = OTHER; note = 2'-O-methylguanine
  modified_base 21: mod_base = OTHER; note = 2'-O-methyluracil
  modified_base 22: mod_base = OTHER; note = 2'-O-methylcytosine
  modified_base 23: mod_base = OTHER; note = 2'-O-methylguanine
  modified_base 1..3: mod_base = OTHER; note = Phosphorothioate bond
  modified_base 21..23: mod_base = OTHER; note = Phosphorothioate bond
ttgtgtttcaactgttctcgtcg

SEQ ID NO: 47 - RNA, 21 nt, synthetic construct
  source 1..21: mol_type = other RNA
  modified_base 1: mod_base = OTHER; note = 2'-O-methyladenine
  modified_base 2: mod_base = OTHER; note = 2'-O-methyluracil
  modified_base 3: mod_base = OTHER; note = 2'-O-methylcytosine
  modified_base 4: mod_base = OTHER; note = 2'-O-methyluracil
  modified_base 5: mod_base = OTHER; note = 2'-O-methyladenine
  modified_base 6: mod_base = OTHER; note = 2'-O-methylguanine
  modified_base 7: mod_base = OTHER; note = 2'-fluorouracil
  modified_base 8: mod_base = OTHER; note = 2'-O-methyladenine
  modified_base 9: mod_base = OTHER; note = 2'-fluoroadenine
  modified_base 10: mod_base = OTHER; note = 2'-fluorouracil
  modified_base 11: mod_base = OTHER; note = 2'-fluoroguanine
  modified_base 12: mod_base = OTHER; note = 2'-O-methyluracil
  modified_base 13: mod_base = OTHER; note = 2'-O-methylcytosine
  modified_base 14: mod_base = OTHER; note = 2'-O-methyluracil
  modified_base 15..16: mod_base = OTHER; note = 2'-O-methyladenine
  modified_base 17: mod_base = OTHER; note = 2'-O-methylguanine
  modified_base 18: mod_base = OTHER; note = 2'-O-methyluracil
  modified_base 19..20: mod_base = OTHER; note = 2'-O-methyladenine
  modified_base 21: mod_base = OTHER; note = 2'-O-methyluracil
  modified_base 1..3: mod_base = OTHER; note = Phosphorothioate bond
  modified_base 19..21: mod_base = OTHER; note = Phosphorothioate bond
atctagtaatgtctaagtaat

SEQ ID NO: 48 - RNA, 21 nt, synthetic construct
  source 1..21: mol_type = other RNA
  modified_base 20..21: mod_base = OTHER; note = Thymine
gaggatatctggaagaaattt

SEQ ID NO: 49 - RNA, 21 nt, synthetic construct
  source 1..21: mol_type = other RNA
  modified_base 20..21: mod_base = OTHER; note = Thymine
atttcttccagatatcctctt

SEQ ID NO: 50 - RNA, 21 nt, synthetic construct
  source 1..21: mol_type = other RNA
  modified_base 20..21: mod_base = OTHER; note = Thymine
gagaacagttgaaacacaatt

SEQ ID NO: 51 - RNA, 21 nt, synthetic construct
  source 1..21: mol_type = other RNA
  modified_base 20..21: mod_base = OTHER; note = Thymine
ttgtgtttcaactgttctctt

SEQ ID NO: 52 - RNA, 21 nt, synthetic construct
  source 1..21: mol_type = other RNA
  modified_base 20..21: mod_base = OTHER; note = Thymine
aacacaaacttgaacagcttt

SEQ ID NO: 53 - RNA, 21 nt, synthetic construct
  source 1..21: mol_type = other RNA
  modified_base 20..21: mod_base = OTHER; note = Thymine
agctgttcaagtttgtgtttt

SEQ ID NO: 54 - RNA, 21 nt, synthetic construct
  source 1..21: mol_type = other RNA
  modified_base 20..21: mod_base = OTHER; note = Thymine
aagaagatgaggaagaaattt

SEQ ID NO: 55 - RNA, 21 nt, synthetic construct
  source 1..21: mol_type = other RNA
  modified_base 20..21: mod_base = OTHER; note = Thymine
atttcttcctcatcttctttt

SEQ ID NO: 56 - RNA, 24 nt, synthetic construct
  source 1..24: mol_type = other RNA
  modified_base 23..24: mod_base = OTHER; note = Thymine
cacagcccactggtcctcaagatt

SEQ ID NO: 57 - RNA, 24 nt, synthetic construct
  source 1..24: mol_type = other RNA
  modified_base 23..24: mod_base = OTHER; note = Thymine
tcttgaggaccagtgggctgtgtt

SEQ ID NO: 58 - RNA, 23 nt, synthetic construct
  source 1..23: mol_type = other RNA
  modified_base 22..23: mod_base = OTHER; note = Thymine
aagaggcgaacacacaacgtctt

SEQ ID NO: 59 - RNA, 23 nt, synthetic construct
  source 1..23: mol_type = other RNA
  modified_base 22..23: mod_base = OTHER; note = Thymine
gacgttgtgtgttcgcctctttt

SEQ ID NO: 60 - RNA, 19 nt, synthetic construct
  source 1..19: mol_type = other RNA
  modified_base 18..19: mod_base = OTHER; note = Thymine
cagatcagcaacaaccgtt

SEQ ID NO: 61 - RNA, 19 nt, synthetic construct
  source 1..19: mol_type = other RNA
  modified_base 18..19: mod_base = OTHER; note = Thymine
cggttgttgctgatctgtt

SEQ ID NO: 62 - RNA, 27 nt, synthetic construct
  source 1..27: mol_type = other RNA
  modified_base 26..27: mod_base = OTHER; note = Thymine
gagaccttcatcaaaaacatcatcatt

SEQ ID NO: 63 - RNA, 27 nt, synthetic construct
  source 1..27: mol_type = other RNA
  modified_base 26..27: mod_base = OTHER; note = Thymine
tgatgatgtttttgatgaaggtctctt

SEQ ID NO: 64 - RNA, 21 nt, synthetic construct
  source 1..21: mol_type = other RNA
  modified_base 20..21: mod_base = OTHER; note = Thymine
gagctaaaacggagctttttt

SEQ ID NO: 65 - RNA, 21 nt, synthetic construct
  source 1..21: mol_type = other RNA
  modified_base 20..21: mod_base = OTHER; note = Thymine
aaaagctccgttttagctctt

SEQ ID NO: 66 - RNA, 21 nt, synthetic construct
  source 1..21: mol_type = other RNA
gcgaggatatctggaagaaat

SEQ ID NO: 67 - RNA, 23 nt, synthetic construct
  source 1..23: mol_type = other RNA
atttcttccagatatcctcgctg

SEQ ID NO: 68 - RNA, 21 nt, synthetic construct
  source 1..21: mol_type = other RNA
acgagaacagttgaaacacaa

SEQ ID NO: 69 - RNA, 23 nt, synthetic construct
  source 1..23: mol_type = other RNA
ttgtgtttcaactgttctcgtcg

SEQ ID NO: 70 - RNA, 21 nt, synthetic construct
  source 1..21: mol_type = other RNA
acaagaagatgaggaagaaat

SEQ ID NO: 71 - RNA, 23 nt, synthetic construct
  source 1..23: mol_type = other RNA
atttcttcctcatcttcttgttc

SEQ ID NO: 72 - RNA, 21 nt, synthetic construct
  source 1..21: mol_type = other RNA
aagaggcgaacacacaacgtc

SEQ ID NO: 73 - RNA, 23 nt, synthetic construct
  source 1..23: mol_type = other RNA
gacgttgtgtgttcgcctcttga

SEQ ID NO: 74 - RNA, 21 nt, synthetic construct
  source 1..21: mol_type = other RNA
gagagctaaaacggagctttt

SEQ ID NO: 75 - RNA, 23 nt, synthetic construct
  source 1..23: mol_type = other RNA
aaaagctccgttttagctctcgt

SEQ ID NO: 76 - RNA, 19 nt, synthetic construct
  source 1..19: mol_type = other RNA
gtctcttggatattctcga

SEQ ID NO: 77 - RNA, 21 nt, synthetic construct
  source 1..21: mol_type = other RNA
tcgagaatatccaagagacag

SEQ ID NO: 78 - RNA, 19 nt, synthetic construct
  source 1..19: mol_type = other RNA
cagctaattcagaatcatt

SEQ ID NO: 79 - RNA, 21 nt, synthetic construct
  source 1..21: mol_type = other RNA
aatgattctgaattagctgta

SEQ ID NO: 80 - DNA, 19 bp, synthetic construct
  source 1..19: mol_type = other DNA
gggcactgtttaaaggaaa

SEQ ID NO: 81 - DNA, 23 bp, synthetic construct
  source 1..23: mol_type = other DNA
ttgaacaagagcaatgactttga

SEQ ID NO: 82 - DNA, 21 bp, synthetic construct
  source 1..21: mol_type = other DNA
aacaagagcaatgactttgag

SEQ ID NO: 83 - DNA, 19 bp, synthetic construct
  source 1..19: mol_type = other DNA
gagcaatgactttgaggaa

SEQ ID NO: 84 - DNA, 21 bp, synthetic construct
  source 1..21: mol_type = other DNA
ggacatctcagacccgtacaa

SEQ ID NO: 85 - DNA, 19 bp, synthetic construct
  source 1..19: mol_type = other DNA
ccaacaaactggagagaga

SEQ ID NO: 86 - DNA, 21 bp, synthetic construct
  source 1..21: mol_type = other DNA
aagctctttgacacacagcag

SEQ ID NO: 87 - DNA, 21 bp, synthetic construct
  source 1..21: mol_type = other DNA
aagctcatcacagctcacgta

SEQ ID NO: 88 - DNA, 25 bp, synthetic construct
  source 1..25: mol_type = other DNA
cctttcagtattgacactgactaga

SEQ ID NO: 89 - DNA, 23 bp, synthetic construct
  source 1..23: mol_type = other DNA
atctagtaatgtctaagtaatgg

SEQ ID NO: 90 - DNA, 21 bp, synthetic construct
  source 1..21: mol_type = other DNA
aagacctccacttgcttaagt

SEQ ID NO: 91 - DNA, 25 bp, synthetic construct
  source 1..25: mol_type = other DNA
gataaagtggaattggcaaactaga